SEQUENCE LISTING (32 sequences)

SEQ ID NO: 1 - DNA, 1523 bp, Homo Sapiens
atttttttggtgtgtgtgtcttcaccgaacattcaaaactgtttctccaaagcgttttgcaaaaactcagactgttttccaaagcagaagcactggagtccccagcagaagcgatgggcagtgtgcgaaccaaccgctacagcatcgtttcttcagaagaagacggtatgaagttggccaccatggcagttgcaaatggctttgggaacgggaagagtaaagtccacacccgacaacagtgcaggagccgctttgtgaagaaagatggccactgtaatgttcagttcatcaatgtgggtgagaaggggcaacggtacctcgcagacatcttcaccacgtgtgtggacattcgctggcggtggatgctggttattttctgcctggctttcgtcctgtcatggctgttttttggctgtgtgttttggttgatagctctgctccatggggacctggatgcatccaaagagggcaaagcttgtgtgtccgaggtcaacagcttcacggctgccttcctcttctccattgagacccagacaaccataggctatggtttcagatgtgtcacggatgaatgcccaattgctgttttcatggtggtgttccagtcaatcgtgggctgcatcatcgatgctttcatcattggcgcagtcatggccaagatggcaaagccaaagaagagaaacgagactcttgtcttcagtcacaatgccgtgattgccatgagagacggcaagctgtgtttgatgtggcgagtgggcaatcttcggaaaagccacttggtggaagctcatgttcgagcacagctcctcaaatccagaattacttctgaaggggagtatatccctctggatcaaatagacatcaatgttgggtttgacagtggaatcgatcgtatatttctggtgtccccaatcactatagtccatgaaatagatgaagacagtcctttatatgatttgagtaaacaggacattgacaacgcagactttgaaatcgtggtcatactggaaggcatggtggaagccactgccatgacgacacagtgccgtagctcttatctagcaaatgaaatcctgtggggccaccgctatgagcctgtgctctttgaagagaagcactactacaaagtggactattccaggttccacaaaacttacgaagtccccaacactcccctttgtagtgccagagacttagcagaaaagaaatatatcctctcaaatgcaaattcattttgctatgaaaatgaagttgccctcacaagcaaagaggaagacgacagtgaaaatggagttccagaaagcactagtacggacacgccccctgacatagaccttcacaaccaggcaagtgtacctctagagcccaggcccttacggcgagagtcggagatatgactgactgattccttctctggaatagttactttacaacacggtctgttggtcagaggcccaaaacagttatacagatgacggtactggtcaagatgggtcaagcaagcggccacacgggactgaggc

SEQ ID NO: 2 - protein, 427 aa, Homo Sapiens
MGSVRTNRYSIVSSEEDGMKLATMAVANGFGNGKSKVHTRQQCRSRFVKKDGHCNVQFINVGEKGQRYLADIFTTCVDIRWRWMLVIFCLAFVLSWLFFGCVFWLIALLHGDLDASKEGKACVSEVNSFTAAFLFSIETQTTIGYGFRCVTDECPIAVFMVVFQSIVGCIIDAFIIGAVMAKMAKPKKRNETLVFSHNAVIAMRDGKLCLMWRVGNLRKSHLVEAHVRAQLLKSRITSEGEYIPLDQIDINVGFDSGIDRIFLVSPITIVHEIDEDSPLYDLSKQDIDNADFEIVVILEGMVEATAMTTQCRSSYLANEILWGHRYEPVLFEEKHYYKVDYSRFHKTYEVPNTPLCSARDLAEKKYILSNANSFCYENEVALTSKEEDDSENGVPESTSTDTPPDIDLHNQASVPLEPRPLRRESEI

SEQ ID NO: 3 - DNA, 21 bp, Homo Sapiens
atgggcagtgtgcgaaccaac

SEQ ID NO: 4 - DNA, 26 bp, Homo Sapiens
tcatatctccgactctcgccgtaagg

SEQ ID NO: 5 - DNA, 21 bp, Homo Sapiens
catgcagtcctccttaccatc

SEQ ID NO: 6 - DNA, 20 bp, Homo Sapiens
aggaagagttcttgcagctc

SEQ ID NO: 7 - DNA, 20 bp, Homo Sapiens
tcctctctggctccatcgta

SEQ ID NO: 8 - DNA, 21 bp, Homo Sapiens
gcaattaaccctcactaaagg

SEQ ID NO: 9 - DNA, 20 bp, Homo Sapiens
tccgaggtcaacagcttcac

SEQ ID NO: 10 - DNA, 20 bp, Homo Sapiens
ttgggcattcatccgtgaca

SEQ ID NO: 11 - DNA, 20 bp, Homo Sapiens
tgatggctggagggtagagg

SEQ ID NO: 12 - DNA, 20 bp, Homo Sapiens
gggtagcttagtagggctgc

SEQ ID NO: 13 - DNA, 25 bp, Homo Sapiens
aaaacaacaactcagtcaaaatgcc

SEQ ID NO: 14 - DNA, 20 bp, Homo Sapiens
tcctcgctgatccagaagtt

SEQ ID NO: 15 - DNA, 23 bp, Homo Sapiens
cagtggcaggagtacaaagacat

SEQ ID NO: 16 - DNA, 24 bp, Homo Sapiens
actgtggagttcatagtcaaggtc

SEQ ID NO: 17 - DNA, 20 bp, Homo Sapiens
cattgcacaaatccgccctg

SEQ ID NO: 18 - DNA, 21 bp, Homo Sapiens
taaaaagggctccctcgacac

SEQ ID NO: 19 - DNA, 20 bp, Homo Sapiens
ccctcaagcatgcaggtaca

SEQ ID NO: 20 - DNA, 20 bp, Homo Sapiens
gcaatcctctgagttgggct

SEQ ID NO: 21 - DNA, 20 bp, Homo Sapiens
gcatgcaagaggagctctga

SEQ ID NO: 22 - DNA, 20 bp, Homo Sapiens
cacaaggcctctcacaccat

SEQ ID NO: 23 - DNA, 20 bp, Homo Sapiens
ctggtaagtggaggtgctgg

SEQ ID NO: 24 - DNA, 20 bp, Homo Sapiens
agtgagctacagcacagcag

SEQ ID NO: 25 - DNA, 20 bp, Homo Sapiens
actgatccagcacgtaagcc

SEQ ID NO: 26 - DNA, 20 bp, Homo Sapiens
atctgtgtgtgccccagaag

SEQ ID NO: 27 - DNA, 20 bp, Homo Sapiens
aggaggggctctgttctcat

SEQ ID NO: 28 - DNA, 20 bp, Homo Sapiens
ctcactcttgctcagcctgg

SEQ ID NO: 29 - DNA, 20 bp, Homo Sapiens
tgcatttcaaggcaaggcag

SEQ ID NO: 30 - DNA, 19 bp, Homo Sapiens
tagggggcttgggactgag

SEQ ID NO: 31 - DNA, 20 bp, Homo Sapiens
tccgaggtcaacagcttcac

SEQ ID NO: 32 - DNA, 20 bp, Homo Sapiens
ttgggcattcatccgtgaca

(19) United States
(12) Patent Application Publication
(10) Pub. No.: US 2019/0376039 A1
(43) Pub. Date: Dec. 12, 2019

(54) CELLS WITH IMPROVED INWARD RECTIFIER CURRENT

(71) Applicants: L. Lee Lochbaum Eckhardt, Madison, WI (US); Ravi Vaidyanathan, Madison, WI (US)

(72) Inventors: L. Lee Lochbaum Eckhardt, Madison, WI (US); Ravi Vaidyanathan, Madison, WI (US)

(21) Appl. No.: 16/433,565

(22) Filed: Jun. 6, 2019

Related U.S. Application Data
(60) Provisional application No. 62/682,631, filed on Jun. 8, 2018.

(51) Int. Cl. C12N 5/077; C12N 15/90; C12N 15/85; G01N 33/50; C07K 14/47

(52) U.S. Cl. CPC C12N 5/0657; C12N 15/907; C12N 15/85; C12N 2800/80; C07K 14/47; C12N 2310/20; G01N 33/502

(57) ABSTRACT

Provided herein is technology relating to differentiated stem cells and particularly, but not exclusively, to stem-cell derived cardiomyocytes having improved inward rectifier currents (e.g., I_{K1} currents), methods for producing stem-cell derived cardiomyocytes having inward rectifier currents, and systems and uses related to stem-cell derived cardiomyocytes having enhanced inward rectifier currents.

This application claims priority to U.S. provisional patent application Ser. No. 62/682,631, filed Jun. 8, 2018, which is incorporated herein by reference in its entirety.

### STATEMENT REGARDING FEDERALLY SPONSORED RESEARCH OR DEVELOPMENT

This technology was made with government support under Grant No. P30 HD03352 awarded by the National Institutes of Health. The government has certain rights in the technology.

### FIELD

Provided herein is technology relating to differentiated stem cells and particularly, but not exclusively, to stem-cell derived cardiomyocytes having improved inward rectifier current (e.g., I_{K1}), methods for producing stem-cell derived cardiomyocytes having inward rectifier currents, and systems and uses related to stem-cell derived cardiomyocytes having enhanced inward rectifier current.

### BACKGROUND

The cardiac action potential (AP) is a change in membrane potential across heart cells that is caused by the movement of ions across the membrane through ion channels. In a healthy heart, the sinoatrial node (SAN) produces approximately 60-100 AP per minute, which causes a heart to beat normally. AP production and AP rate are fundamental biological properties of cardiac cells, which are often measured using an electrocardiogram (ECG). An ECG comprises characteristic upward and downward peaks (P, Q, R, S and T) that represent the depolarization (voltage becoming more positive) and repolarization (voltage becoming more negative) of the action potential in the atria and ventricles. Repolarization of the AP is often characterized by the time between the start of the Q peak and the end of the T peak in the heart's electrical cycle, which is termed the QT interval. Thus, a lengthened QT interval indicates anomalies in repolarization and is associated with potentially fatial ventricular tachyarrhythmias like torsades de pointes (TdP) and is a risk factor for sudden death.

In addition to genetic causes of AP anomalies, some drugs also affect generation of AP in the heart. Consequently, drug-induced cardiac action potential (AP) prolongation (e.g., as demonstrated by a long QT interval) can cause arrhythmias and fatality. Although originally described for antiarrhythmic drugs such as quinidine and sotalol, drug-induced long QT syndrome (di-LQTS) occurs with both cardiac and non-cardiovascular medications.

Pre-clinical cardiac safety of new pharmacologic agents, as implemented by the International Council for Harmonization of Technical Requirements for Pharmaceuticals for Human Use (ICH) guidelines for clinical (E14) and non-clinical drug development (S7B), requires arrhythmia risk assessment by determination of hERG (Kv11.1) channel (rapidly activating delayed rectifier potassium current, I_{Kr}) block,

Some ion channels (e.g., hERG channels) are vulnerable to drugs that bind to and block ion transport through the channels, resulting in AP prolongation and triggering arrhythmogenic early afterdepolarizations (EADs) (1). Consequently, pre-clinical cardiac safety evaluation of pharmacologic agents, e.g., as implemented by the International Council for Harmonization of Technical Requirements for Pharmaceuticals for Human Use (ICH) guidelines for clinical (E14) and non-clinical drug development (S7B), requires arrhythmia risk assessment. In particular, pre-clinical cardiac safety assessment involves determining if pharmacologic agents block hERG channels (Kv11.1) and the associated rapidly activating delayed rectifier potassium current (I_{Kr}).

While assessing hERG channel blockade by drugs provides a component of evaluating the safety of drugs, it is well established that other ion channels are affected by drugs that cause changes in the underlying cardiac AP and, e.g., can cause di-LQTS and arrhythmia susceptibility. Additionally, testing for hERG block in vitro does not mimic effects of drugs on human cardiac tissue or its AP in vivo. Thus, a consequence of the ICH S7B and E14 policy is that some harmful drugs have reached clinical use and other drugs have been excluded from use that have low arrhythmogenic risk (2). Further, nearly 50% of drugs removed from the market are removed due to cardiovascular complications and arrhythmias predisposing to sudden cardiac death (3).

One response to address this issue is the Comprehensive in vitro Proarrhythmia Assay (CIPA), which more robust safety data for pre-clinical studies. Stem cell derived-cardiomyocytes (SC-CM) are included in the CIPA model as a reproducible source of human cardiac myocytes. However, SC-CM are widely criticized in both the research and pharmaceutical communities because they exhibit immature cellular phenotypes (4) and are not able to sufficiently model ventricular arrhythmic vulnerability. Compared to adult human cardiac myocytes, SC-CM have a relatively depolarized resting membrane potential and spontaneous automaticity due to a small and insufficient inward rectifier current (I_{K1}) density and unopposed pacemaker current (I_{f}) (5, 6). Recorded I_{K1} from available SC-CM lines is either absent or too small to be considered physiologic (5). The lack of a normally polarized resting membrane potential causes partial inactivation of ion channels including the cardiac sodium channel and reduces its current (I_{Na}) amplitude (7). Automaticity also interferes with electrical pacing and the ability to generate APs at slow rates. This is particularly problematic in the study of the arrhythmia torsade de points, the signature arrhythmia related long QT syndrome. AP prolongation creates vulnerability for triggered activity of early after-depolarizations (EADs), which is the triggered cellular event required to induce torsade de points. EADs occur at low stimulation frequencies and are suppressed with increased pacing rates. Thus, since torsades de pointes is EAD driven, it has bradycardic- and pause-dependent arrhythmia induction and control of SC-CM frequency is imperative to model this arrhythmia (8, 1).

Various solutions to compensate for the small or insufficient I_{K1} in SC-CM have been attempted including adenoviral infection/overexpression (9-11) of the KCNJ2 gene encoding the potassium inward rectifier channel Kir2.1 or by cellular current injection (12) to augment the I_{K1} rectifying current. However, adenoviral infection and cellular current injection are not conducive to long term cell culture maintenance. Additionally, current injection inadequately addresses the other cellular influences on transcription that occur with Kir2.1 functional expression (9, 13). Accordingly, new technologies are needed.

### SUMMARY

Accordingly, embodiments of the technology described herein provide a stem cell line (14) modified using gene editing to comprise an enhanced I_{K1} inward rectifier current density. In some embodiments, the modified stem cells provide a reliable, reproducible SC-CM platform for studying adult-like cardiac APs and for drug safety testing. During experiments conducted during the development of embodiments of the technology described herein, SC-CMs enhanced with I_{K1} generated stable resting membrane potentials without spontaneous automaticity, had increased cell capacitance, and increased rates of AP upstroke (increased dV/dT) consistent with normal activation of I_{Na}. Importantly, the SC-CM enhanced with I_{K1} have AP characteristics comparable to adult ventricular myocytes and could be stimulated over a wide range of pacing rates.

Provided herein is technology relating to differentiated stem cells and particularly, but not exclusively, to stem-cell derived cells (e.g., cardiomyocytes (e.g., cardiomyocytes produced from differentiated stem cells)) having inward rectifier currents (e.g., I_{K1} currents). In some embodiments, cardiomyocytes have inward rectifier currents characteristic of an adult and/or mature and/or healthy and/or normal cardiomyocytes. In some embodiments, the technology relates to stem-cell derived cardiomyocytes (e.g., cardiomyocytes produced from differentiated stem cells) having improved inward rectifier currents (e.g., I_{K1} currents). Some embodiments of the technology provide methods for production of stem-cell derived cardiomyocytes having inward rectifier currents (e.g., I_{K1} currents) or improved inward rectifier currents (e.g., I_{K1} currents). Some embodiments provide systems and uses related to stem-cell derived cardiomyocytes having inward rectifier currents (e.g., I_{K1} currents) or improved inward rectifier currents (e.g., I_{K1} currents).

Accordingly, in some embodiments the technology provides a stem cell derived cardiomyocyte comprising a nucleic acid encoding an inducible potassium inward rectifier channel. In some embodiments, the stem cell derived cardiomyocyte comprises a nucleic acid comprising a Kir sequence. In some embodiments, the stem cell derived cardiomyocyte comprises a nucleic acid comprising a Kir2 sequence. In some embodiments, the stem cell derived cardiomyocyte comprises a nucleic acid comprising a Kir2.1 sequence. In some embodiments, the stem cell derived cardiomyocyte comprises a nucleic acid comprising a Kir2.1 cDNA or genomic sequence. Embodiments relate to nucleic acid constructs comprising a potassium inward rectifier channel operably linked to an inducible promoter. For instance, in some embodiments the technology provides a nucleic acid comprising an inducible promoter operably linked to a nucleic acid encoding a Kir2.1. In some embodiments, the technology provides a doxycycline-inducible promoter operably linked to a nucleic acid encoding a Kir2.1. In some embodiments, the cell derived cardiomyocyte comprises a TRE3G promoter operably linked to a nucleic acid encoding a Kir2.1. In some embodiments, the technology provides a stem cell derived cardiomyocyte comprising a sequence from KCNJ2. In some embodiments, the technology provides a stem cell derived cardiomyocyte comprising a sequence that is at least 80% identical to KCNJ2. In some embodiments, the technology provides a stem cell derived cardiomyocyte comprising a sequence that is at least 90% identical to KCNJ2. In some embodiments, the technology provides a stem cell derived cardiomyocyte comprising a sequence that is at least 95% identical to KCNJ2. In some embodiments, the technology provides a stem cell derived cardiomyocyte comprising a sequence that is at least 99% identical to KCNJ2. In some embodiments, the technology provides a stem cell derived cardiomyocyte comprising a sequence that is 100% identical to KCNJ2. In some embodiments, the technology provides a stem cell derived cardiomyocyte comprising a sequence that is at least 75, 80, 85, 90, 91, 92, 93, 94, 95, 96, 97, 98, 99, 99.5, or 99.9% identical to KCNJ2.

Embodiments of the technology provide a stem cell derived cardiomyocyte comprising an inducible potassium inward rectifier current (I_{K1}).

Embodiments of the technology relate to methods. For example, in some embodiments, the technology provides a method of producing a physiologically mature stem cell derived cardiomyocyte, the method comprising providing a stem cell derived cardiomyocyte comprising a nucleic acid encoding an inducible potassium inward rectifier channel; and inducing expression of said inducible potassium inward rectifier channel in said stem cell derived cardiomyocyte. In some embodiments, methods further comprise pacing said stem cell derived cardiomyocyte. In some embodiments, inducing the stem cell derived cardiomyocyte comprising a nucleic acid encoding an inducible potassium inward rectifier channel comprises contacting said stem cell derived cardiomyocyte comprising a nucleic acid encoding an inducible potassium inward rectifier channel with a composition comprising an inducer (e.g., a compound that activates the promoter). In some embodiments, providing the stem cell derived cardiomyocyte comprising a nucleic acid encoding an inducible potassium inward rectifier channel comprises thawing a stem cell derived cardiomyocyte comprising a nucleic acid encoding an inducible potassium inward rectifier channel from a stored preparation. In some embodiments, providing the stem cell derived cardiomyocyte comprising a nucleic acid encoding an inducible potassium inward rectifier channel comprises constructing said cardiomyocyte comprising a nucleic acid encoding an inducible potassium inward rectifier channel using a CRISPR technology.

Additional embodiments relate to systems. For instance, in some embodiments, the technology provides a system for testing the cardiac safety of a drug, the system comprising a stem cell derived cardiomyocyte expressing a potassium inward rectifier channel; and a cellular electrophysiology measurement system. In some embodiments the system further comprises the drug (e.g., the drug to be tested for cardiac safety). In some embodiments, the systems further comprise an inducer composition for inducing expression of said potassium inward rectifier channel in said stem cell derived cardiomyocyte. In some embodiments, the stem cell derived cardiomyocyte has a physiologically mature phenotype. In some embodiments, the systems further comprise a component to pace said stem cell derived cardiomyocyte.

Additional embodiments relate to a cell expressing an inducible potassium inward rectifier channel. In some embodiments, the cell is a muscle cell or a neurocyte. In some embodiments, the cell is a differentiated stem cell.

Compositions, in some embodiments, comprise the cells described herein. In some embodiments, the technology provides a composition comprising a cell expressing an inducible potassium inward rectifier channel. In some embodiments, the composition of further comprises a test compound. In some embodiments, the composition further comprises an inducing compound.

Some embodiments provide a method for testing a compound for cardiac safety. For instance, in some embodiments, methods comprise providing a physiologically mature stem cell derived cardiomyocyte comprising a nucleic acid encoding an inducible potassium inward rectifier channel; contacting said physiologically mature stem cell derived cardiomyocyte with a test compound; and measuring a physiological phenotype of said physiologically mature stem cell derived cardiomyocyte. In some embodiments, the methods comprise measuring a physiological phenotype that is an action potential (AP), AP amplitude, resting membrane potential, AP duration at 10% of repolarization (APD10), AP duration at 50% of repolarization (APD50), AP duration at 70% of repolarization (APD70), AP duration at 90% of repolarization (APD90) of repolarization, or maximum upstroke velocity (dV/dtmax). Some embodiments further comprise comparing the physiological phenotype of said physiologically mature stem cell derived cardiomyocyte in the presence and absence of said test compound. In some embodiments, the physiologically mature stem cell derived cardiomyocyte has a potassium inward rectifier current similar to a cardiomyocyte in vivo.

Additional embodiments will be apparent to persons skilled in the relevant art based on the teachings contained herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

These and other features, aspects, and advantages of the present technology will become better understood with regard to the following drawings:

FIG. 1 is a series of fluorescence microscope images of I_{K1}-induced SC-CM (bottom panels) and non-induced SC-CM (top panels) immunostained for Kir2.1, cardiac troponin T (cTnT), and myosin light chain 2a (MLC2a). Merged panels are on the far right. Scale bar=25 μm.

FIG. 2 is a plot of I_{K1} recorded for induced SC-CM (grey line) and non-induced SC-CM (black line). Summary current density was recorded from the SC-CMs using a voltage ramp protocol as shown in the inset. * denotes p<0.05.

FIG. 3A-FIG. 3D show AP characteristics from I_{K1}-induced ventricular-like SC-CMs. FIG. 3A is a plot showing representative AP from ventricular-like I_{K1}-induced SC-CMs when paced at 0.5 Hz, 1 Hz, 2 Hz, and 3 Hz. The scale bar is applicable to all APs in Figure FIG. 3A. FIG. 3B is a bar plot showing APD for I_{K1}-induced ventricular-like SC-CMs calculated at 10% repolarization (APD₁₀, black), 50% repolarization (APD50, right-up hashing (/////)), 70% repolarization (APD70, right-down hashing (\\\\\)), and 90% repolarization (APD90, horizontal hashing) from peak at pacing frequencies of 0.5 Hz, 1 Hz, 2 Hz, and 3 Hz. FIG. 3C is a bar plot showing RMP of I_{K1}-induced ventricular-like SC-CMs at pacing frequencies of 0.5 Hz, 1 Hz, 2 Hz and 3 Hz. FIG. 3D is a bar plot showing maximum upstroke velocity (dV/dtmax) for I_{K1}-induced ventricular-like SC-CMs at pacing frequencies of 0.5 Hz, 1 Hz, 2 Hz, and 3 Hz. FIG. 3B, FIG. 3C, and FIG. 3D show data from experiments testing the same n=number of cells. * denotes p<0.01.

FIG. 4A-FIG. 4D show data indicating that E4031 prolongs APD₇₀ and APD₉₀ in I_{K1}-induced ventricular-like SC-CMs. FIG. 4A shows representative APs from ventricular-like I_{K1}-induced SC-CMs in the absence of E4031 (non-treated control, black line) and the same ventricular-like I_{K1}-induced SC-CMs when perfused with E4031 (grey line). FIG. 4B is a bar plot showing normalized data (as a percentage) at a 0.5 Hz pacing frequency (n=6). FIG. 4C is a scatter plot of data recording the take-off potential versus peak voltage calculated from the early after depolarizations generated when I_{K1}-induced SC-CMs were treated with 100 nM E4031. FIG. 4D is a scatter plot of data from a second, independent experiment recording the take-off potential versus peak voltage calculated from the early after depolarizations generated when I_{K1}-induced SC-CMs were treated with 100 nM E4031. The data in FIG. 4C and FIG. 4D are fit with a linear regression model. * denotes p<0.05.

FIG. 5A and FIG. 5B show that ATX-II prolongs APD₅₀, APD₇₀, and APD₉₀ in I_{K1}-induced ventricular-like SC-CMs. FIG. 5A shows representative AP from ventricular-like I_{K1}-induced SC-CMs in the absence of ATX-II (non-treated control, black line) and the same ventricular-like I_{K1}-induced SC-CMs when perfused with ATX-II (grey line). FIG. 5B shows APD analysis for APD₁₀ (black), APD50 (right-up hashing (/////)), APD₇₀ (right-down hashing (\\\\\)), and APD₉₀ (horizontal hashing) at 0.5 Hz pacing frequency under untreated control conditions (left) and when perfused with ATX-II (right) (n=8). # denotes p<0.05, * denotes p<0.01.

FIG. 6A, FIG. 6B, and FIG. 6C show that I_{K1}-induced hiPSC-CMs have robust cardiac protein expression and high purity. FIG. 6A is a schematic drawing showing a method for differentiation, purification, and doxycycline induction of hiPSC-CMs. FIG. 6B shows flow cytometry data indicating that the hPSC-CMs described herein are highly pure: at least 85% of cells are detected to have both cTnT and MLC2a expression. FIG. 6C is a western blot showing robust Kir2.1 protein expression following doxycycline induction (2 μg/ml doxycycline for 48 hours). Kir2.1 was not detected in the absence of doxycycline induction. Beta actin is included as a loading control.

FIG. 7A, FIG. 7B, and FIG. 7C show I_{K1} measured in induced iPS-CMs and non-induced hiPSC-CMs described herein. FIG. 7A shows current traces of non-induced iPS-CM cells (flat trace near 0 pA/pF) and iPS-CM cells induced with 2 μg/ml of doxycycline to express I_{K1} (curved trace). FIG. 7B shows a summary current-voltage relationship using a step protocol (inset) for iPS-CMs that were non-induced (black flat trace) and induced with 2 μg/ml doxycycline. FIG. 7C is a bar graph of the outward current measured at −60 mV for iPS-CMs in the presence of 0 μg/ml (left bar) and 2 μg/ml doxycycline (right bar) (p=0.05).

FIG. 8 is a bar graph of quantitative PCR data indicating increased expression of Kir2.1/KCNJ2 mRNA by hiPSC-CM cells comprising an inducible Kir2.1 in the presence of doxycycline.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to differentiated stem cells and particularly, but not exclusively, to stem-cell derived cardiomyocytes having improved inward rectifier currents (e.g., I_{K1} currents), methods for producing stem-cell derived cardiomyocytes having inward rectifier currents, and systems and uses related to stem-cell derived cardiomyocytes having enhanced inward rectifier currents.

In some embodiments, the technology comprises I_{K1}-inducible SC-CMs that enhance I_{K1} density and respond physiologically to QT prolonging drugs. In some embodiments, the I_{K1}-enhanced SC-CMs develop an I_{K1} density similar to that found in human and vertebrate cardiac myocytes, which is reflected in their capability to repolarize to a normal resting membrane potential without spontaneous automaticity and their capability for pacing response over a wide frequency range. The SC-CMs thus have a more adult-like cardiac AP phenotype that is stable in long-term cell culture. Accordingly, the technology described herein provides a significant advance in the electrophysiology of SC-CMs.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the technology may be readily combined, without departing from the scope or spirit of the technology.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “sequencing-free” method does not comprise a sequencing step, etc.

As used herein, the term “electrophysiology” refers to the electrical properties of a cell or tissue. These electrical properties are measurements of voltage change or electrical current flow at a variety of scales including, but are not limited to, single ion channel proteins, single cells, small populations of cells, tissues comprised of various cell populations, and whole organs (e.g., the heart). Several cell types and tissues that have electrical properties include but are not limited to muscle cells (e.g., heart cells (e.g., cardiomyocytes (e.g., atrial cardiomyocytes (e.g., atrial-like cardiomyocytes), ventricular cardiomyocytes (e.g., ventricular-like cardiomyocytes)))), liver cells, pancreatic cells, ocular cells, and neuronal cells. The electrical properties of a cell or tissue can be measured by the use of electrodes (examples include, but are not limited to, simple solid conductors including discs and needles, tracings on printed circuit boards, and hollow tubes, such as glass pipettes, filled with an electrolyte). Intracellular recordings can be made using techniques such as voltage clamp, current clamp, patch-clamp, or sharp electrode methods. Extracellular recordings can be made using techniques such as single unit recording, field potentials, and amperometry methods. A technique for high throughput analysis can also be used, such as the planar patch clamp. In another aspect, the Bioelectric Recognition Assay (BERA) can be used to measure changes in the membrane potential of cells. Exemplary techniques are described in, e.g., U.S. Pat. Nos. 7,270,730; 5,993,778; and 6,461,860, and are described in Hamill et al. (1981) Pflugers Arch. 391(2)85-100; Alvarez et al. (2002) Adv. Physiol. Educ. 26(1-4)327-341; Kornreich (2007) J. Vet. Cardiol. 9(1)25-37; Perkins (2006) J. Neurosci. Methods. 154(1-2)1-18; Gurney (2000) J. Pharmacol. Toxicol. Methods. 44(22)409-420; Baker et al. (1999) J. Neurosci. Methods 94(1)5-17; McNames and Pearson (2006) Conf. Proc. IEEE Eng. Med. Biol. Soc. 1(1): 1185-1188; Porterfield (2007) Biosens. Bioelectron. 22(7):1186-1196; Wang and Li (2003) Assay Drug Dev. Technol. 1(5)695-708; and Kintzios et al. (2001) Biosens. Bioelectron. 16(4-5)325-336, each of which is included herein by reference. In addition to the electrophysiology of a cell or tissue being measured by the techniques described above, the electrophysiology of larger organs can be measured by additional techniques such as, e.g., an electrocardiogram (ECG or EKG). An ECG records the electrical activity of the heart over time. Analysis of the depolarization and repolarization waves results a description of the electrophysiology of the total heart muscle.

As used herein, the term “phenotype” refers to a description of an individual's trait or characteristic that is measurable and that is sometimes expressed only in a subset of individuals within a population. In one aspect of the technology, an individual's phenotype includes the phenotype of a single cell, a substantially homogeneous population of cells, a population of differentiated cells, or a tissue comprised of a population of cells.

As used herein, the term “electrophysiological phenotype” of a cell or tissue refers to the measurement of a cell or tissue's action potential (“AP”). An action potential is a spike of electrical discharge that travels along the membrane of a cell. The properties of action potentials differ depending on the cell type or tissue. For example, cardiac action potentials are significantly different from the action potentials of most neuronal cells. In one embodiment, the action potential is a cardiac action potential. The “cardiac action potential” is a specialized action potential in the heart, with unique properties necessary for function of the electrical conduction system of the heart. The cardiac action potential has 5 phases; phase 4 (resting membrane potential), phase 0 (rapid depolarization), phase 1 (inactivation of the fast Na+ channels causing a small downward deflection of the action potential), phase 2 (plateau phase—the sustained balance between inward movement of Ca2+ and outward movement of K+), phase 3 (cell repolarization), and back to phase 4. The cardiac action potentials of cells comprising the different portions of the heart have unique features and patterns specific to those cells including, atrial, ventricular, and pacemaker action potentials.

As used herein, the term “pacing” refers to the regulation of contraction of heart muscle, cardiomyocytes, or other heart cells by the application of electrical stimulation pulses or shocks to the heart muscle, cardiomyocytes, or other heart cells. Exemplary methods for pacing cells and/or groups of cells include, but are not limited to, proving an external current, field stimulation, and optogenetics.

As used herein, the term “I_{K1}” refers to the activity of a cell that results in the inward rectifier current of the cell. It is contemplated that the I_{K1} is a stabilizer of a cell's resting membrane potential. This activity is controlled by a family of proteins termed the inward-rectifier potassium ion channels (Kir channels). There are seven subfamilies of Kir channels (Kir1, Kir2, Kir3, Kir4, Kir5, Kir6, and Kir7). Each subfamily has multiple members (e.g., Kir2.1, Kir2.2, Kir2.3, etc.). The Kir2 subclass has four members, Kir2.1, Kir2.2, Kir2.3, and Kir2.4. The active Kir channels are formed from homotetrameric membrane proteins. Additionally, heterotetramers can form between members of the same subfamily (e.g., Kir2.1 and Kir2.3) when the channels are overexpressed. The proteins Kir2.1, Kir2.2, Kir2.3, and Kir2.4 are also known as IR_{K1}, IRK2, IRK3, and IRK4, respectively. These proteins have been sequenced and characterized; see, e.g., GenBank Accession Nos. AAF73241, AAF73242, BAC02718, NP_000882, BAD23901, NP_066292, AAL89708, P63252, P52185, P52190, 019182, 018839, Q64273, P49656, P35561, CAA56622, AAY53910, Q14500, P52188, P52187, NP_001019861, NP_690607, NP_609903, Q64198, P52189, NP_004972, AAF97619, NP_733838, Q8JZN3, and O70596, each of which is incorporated herein by reference. The genes for these proteins have been sequenced and characterized; see, e.g., GenBank Accession Nos. AB074970, AF153819, NM_000891, AB182123, NM_021012, AF482710, X80417, DQ023214, NM_001024690, NM_152868, NM_004981, AF181988, and NM_170720, each of which is incorporated herein by reference.

As used herein, the term “I_{f}” refers the activity of a cell that results in the “funny” or pacemaker current of the cell. It is contemplated that this current functionally modulates pacing of cells that compose the heart (e.g., the cells that compose the SA node). The I_{f} activity is a mixed Na+/K+ inward current activated by hyperpolarization and modulated by the autonomic nervous system. This activity is controlled by a family of proteins termed the hyperpolarization-activated cyclic-nucleotide-modulated channels (HCN channels). There are four members of the HCN family (e.g. HCN1, HCN2, HCN3, and HCN4). HCN isoforms have been shown to coassemble and form heteromultimers. An HCN channel is activated by membrane hyperpolarization and modulated by cAMP and cGMP. These proteins have been sequenced and characterized; see, e.g., GenBank Accession Nos. AAO49470, AAO49469, NPH446136, Q9UL51, NP_001185, NP_005468, NP_065948, EDL89402, NP_445827, NP_001034410 and NP_066550, each of which is incorporated herein by reference. The genes for these proteins have been sequenced and characterized, see for example GenBank Accession Nos. AF488550, AF488549, NM_053684, NM_001194, NM_005477, NM_020897, CH474029, and NM_001039321, each of which is incorporated herein by reference.

As used herein, the term “express” refers to the production of a gene product.

As used herein, the term “expression” refers to the process by which polynucleotides are transcribed (e.g., into mRNA or a functional RNA) and/or the process by which a transcribed mRNA is subsequently translated into peptides, polypeptides, or proteins. If the polynucleotide is derived from genomic DNA, expression may include splicing of the mRNA in a eukaryotic cell. “Differentially expressed” as applied to a gene refers to the differential production of the mRNA transcribed from the gene or the protein product encoded by the gene. A differentially expressed gene may be overexpressed or underexpressed (a.k.a. inhibited) as compared to the expression level of a normal or control cell. In one aspect, it refers to overexpression that is 1.5 times, or alternatively, 2 times, or alternatively, at least 2.5 times, or alternatively, at least 3.0 times, or alternatively, at least 3.5 times, or alternatively, at least 4.0 times, or alternatively, at least 5 times, or alternatively 10 times higher or lower than the expression level detected in a control sample. The term “differentially expressed” also refers to nucleotide sequences in a cell or tissue that are expressed where silent in a control cell or not expressed where expressed in a control cell.

As used herein, the term “operably linked” indicates that a nucleic acid (e.g., a gene, a cDNA, etc.) to be expressed is functionally linked to a control sequence (e.g., a promoter, enhancer, transcriptional control sequences, etc.) so that the nucleic acid is properly expressed. Accordingly, the term “operably linked” to a promoter, as used herein, means that the transcription of a nucleic acid is driven and/or regulated by that promoter. A person skilled in the art will understand that being operably linked to a promoter means, in some embodiments, that the promoter is positioned upstream (e.g., at the 5′-end) of the operably linked nucleic acid. The distance to the operably linked nucleic acid may be variable, as long as the promoter of the present invention is capable of driving and/or regulating the transcription of the operably linked nucleic acid. For example, between the promoter and the operably linked nucleic acid, there might be a cloning site, an adaptor, a transcription or translation enhancer, etc. The operably linked nucleic acid may be any coding or non-coding nucleic acid. The operably linked nucleic acid may be in the sense or in the anti-sense direction. Typically, in the case of genetic engineering of host cells, the operably linked nucleic acid is to be introduced into the host cell and is intended to change the phenotype of the host cell. Alternatively, the operably linked nucleic acid is an endogenous nucleic acid from the host cell.

As used herein, the term “genomic locus” or “locus” (plural “loci”) is the specific location of a gene or DNA sequence on a chromosome.

The term “gene” refers to a DNA sequence that comprises control and coding sequences necessary for the production of an RNA having a non-coding function (e.g., a ribosomal or transfer RNA), a polypeptide, or a precursor. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this technology it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein the term “variant” should be taken to mean the exhibition of qualities that have a pattern that deviates from what occurs in nature.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The term “oligonucleotide” as used herein is defined as a molecule comprising two or more deoxyribonucleotides or ribonucleotides, preferably at least 5 nucleotides, more preferably at least approximately 10 to 15 nucleotides and more preferably at least approximately 15 to 50 nucleotides (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more nucleotides). The exact size will depend on many factors, which in turn depend on the ultimate function or use of the oligonucleotide. The oligonucleotide may be generated in any manner, including chemical synthesis, DNA replication, reverse transcription, PCR, or a combination thereof. An oligonucleotide used for nucleic acid amplification (e.g., PCR) is often called a “primer”.

As used herein, the term “gene product” or alternatively a “gene expression product” refers to the polymer of ribonucleotides (e.g., an mRNA, a functional RNA) generated when a gene is transcribed or polymer of amino acids (e.g., peptide or polypeptide) generated when a gene is transcribed and translated.

As used herein, the term “under transcriptional control” indicates that transcription of a polynucleotide sequence, usually a DNA sequence, depends on its being operatively linked to an element which contributes to the initiation of, or promotes, transcription (e.g., a “promoter”). “Operatively linked” intends the polynucleotides are arranged in a manner that allows them to function in a cell.

The terms “protein” and “polypeptide” refer to compounds comprising amino acids joined via peptide bonds and are used interchangeably. The terms “peptide” and “polypeptide” and “protein” are used interchangeably herein, and refer to a polymeric form of amino acids of any length, which can include coded and non-coded amino acids, chemically or biochemically modified or derivatized amino acids, and polypeptides having modified peptide backbones. Conventional one and three-letter amino acid codes are used herein as follows—Alanine: Ala, A; Arginine: Arg, R; Asp aragine: Asn, N; Asp artate: Asp, D; Cysteine: Cys, C; Glutamate: Glu, E; Glutamine: Gln, Q; Glycine: Gly, G; Histidine: His, H; Isoleucine: Ile, I; Leucine: Leu, L; Lysine: Lys, K; Methionine: Met, M; Phenylalanine: Phe, F; Proline: Pro, P; Serine: Ser, S; Threonine: Thr, T; Tryptophan: Trp, W; Tyrosine: Tyr, Y; Valine: Val, V. As used herein, the codes Xaa and X refer to any amino acid.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino, locked nucleic acid (LNA), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogues”); further, the term “nucleic acid sequence” as used herein refers to an oligonucleotide, nucleotide or polynucleotide, and fragments or portions thereof, and to DNA or RNA of genomic or synthetic origin, which may be single or double stranded, and represent the sense or antisense strand.

It is well known that DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. It is also known that all of these 5 types of nucleotides specifically bind to one another in combinations called complementary base pairing. That is, adenine (A) pairs with thymine (T) (in the case of RNA, however, adenine (A) pairs with uracil (U)), and cytosine (C) pairs with guanine (G), so that each of these base pairs forms a double strand. Degenerate codes for nucleotides are: R (G or A), Y (T/U or C), M (A or C), K (G or T/U), S (G or C), W (A or T/U), B (G or C or T/U), D (A or G or T/U), H (A or C or T/U), V (A or G or C), or N (A or G or C or T/U), gap (−).

As used herein, “CRISPR system” refers collectively to transcripts and other elements involved in the expression of and/or directing the activity of CRISPR-associated (“Cas”) genes, including sequences encoding a Cas gene, a Cas nickase, a tracr (trans-activating CRISPR) sequence (e.g., tracrRNA or an active partial tracrRNA), a cr (CRISPR) sequence (e.g., crRNA or an active partial crRNA), or other sequences and transcripts from a CRISPR locus. In embodiments of the technology, the terms guide sequence and guide RNA (gRNA) are used interchangeably. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR RNP complex (e.g., in vitro or in vivo) and direct it to the site of a target sequence in a cell (e.g., after introduction of the RNP).

In some embodiments, a CRISPR system comprises a DNA-targeting RNA comprising two separate RNA molecules (e.g., two RNA polynucleotides, e.g., an “activator-RNA” and a “targeter-RNA”) and is referred to herein as a “double-molecule DNA-targeting RNA” or a “two-molecule DNA-targeting RNA” or a “double guide RNA” or a “dgRNA”. In other embodiments, a CRISPR system comprises a DNA-targeting RNA comprising a single RNA molecule (e.g., a single RNA polynucleotide) and is referred to herein as a “single-molecule DNA-targeting RNA,” a “single guide RNA,” or an “sgRNA.” The term “DNA-targeting RNA” or “guide RNA” or “gRNA” is inclusive, referring both to double-molecule DNA-targeting RNAs (dgRNAs) and to single-molecule DNA-targeting RNAs (sgRNAs).

As used herein, “stem cell” defines a cell with the ability to divide for indefinite periods in culture and give rise to specialized cells. At this time and for convenience, stem cells are categorized as somatic (adult) or embryonic. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. An embryonic stem cell is one that has been cultured under in vitro conditions that allow proliferation without differentiation for months to years. Pluripotent embryonic stem cells can be distinguished from other types of cells by the use of marker including, but not limited to, Oct-4, alkaline phosphatase, CD30, TDGF-1, GCTM-2, Genesis, Germ cell nuclear factor, SSEA1, SSEA3, and SSEA4. The term “stem cell” also includes “dedifferentiated” stem cells, an example of which is a somatic cell which is directly converted to a stem cell (“reprogrammed”). A clone is a line of cells that is genetically identical to the originating cell; in this case, a stem cell.

Adult stem cells, as used in accordance with the present technology, encompass cells that are derived from any adult tissue or organ that replicate as undifferentiated cells and have the potential to differentiate into at least one, preferably multiple, cell lineages. General methods for producing and culturing populations of adult stem cells suitable for use in the present technology are described in WO2006/110806 to Xu et al., WO2002/057430 to Escoms et al., and WO2006/112365 to Nagaya, each of which is incorporated herein in its entirety. Cardiac progenitor or adult stem cells are particularly suitable for use in the present technology. Methods for isolating and culturing cardiac stem cells are described in WO2007/100530, WO2002/009650, and WO2002/013760 all to Anversa; WO2004/019767 to Schneider; and WO2006/052925 to Marban et al., which are all hereby incorporated by reference in their entirety.

In some embodiments, the technology comprises use of embryonic stem cells. Embryonic stem (“ES”) cells include any multi- or pluripotent stem cell derived from pre-embryonic, embryonic, or fetal tissue at any time after fertilization, and have the characteristic of being capable under appropriate conditions of producing progeny of several different cell types that are derivatives of all of the three germinal layers (endoderm, mesoderm, and ectoderm), according to a standard art accepted test (e.g., the ability to form a teratoma in 8-12 week old SCID mice). In some embodiments, the stem cells are mammalian embryonic stem cells. In some embodiments, the embryonic stem cells of the present technology are human embryonic stem cells. Methods for culturing embryonic stems cells, particularly human embryonic stem cells, are known in the art and described in WO2006/029297, WO2006/019366 and WO2006/029198 all to Thomson and Ludwig, and WO2008/089351 to Bergendahl and Thomson, each of which is incorporated herein by reference.

In some embodiments, the technology comprises use of embryonic germ cells. Embryonic germ (“EG”) cells are derived from primordial germ cells and exhibit an embryonic pluripotent cell phenotype. EG cells are capable of differentiation into cells of ectodermal, endodermal, and mesodermal germ layers. EG cells can also be characterized by the presence or absence of markers associated with specific epitope sites. Methods for isolating, culturing, and characterizing human EG cells are described in Shamblott et al., “Human Embryonic Germ Cell Derivatives Express a Broad Range of Developmentally Distinct Markers and Proliferate Extensively In Vitro,” Proc Natl Acad Sci 98(1)113-118 (2001), each of which is incorporated by reference in its entirety.

Adult stem cells, as used in accordance with the present technology, encompass cells that are derived from any adult tissue or organ that replicate as undifferentiated cells and have the potential to differentiate into at least one, preferably multiple, cell lineages. General methods for producing and culturing populations of adult stem cells suitable for use in the present technology are described in WO2006/110806 to Xu et al., WO2002/057430 to Escoms et al., and WO2006/112365 to Nagaya, each of which is incorporated herein by reference. Cardiac progenitor or adult stem cells are particularly suitable for use in the present technology. Methods for isolating and culturing cardiac stem cells are described in WO2007/100530, WO2002/009650, and WO2002/013760 all to Anversa; WO2004/019767 to Schneider; and WO2006/052925 to Marban et al., each of which is incorporated herein by reference.

In some embodiments, the technology comprises use of induced pluripotent stem cells (“iPSC”). iPSCs, as used herein, refer to pluripotent stem cells induced from somatic cells, e.g., a population of differentiated somatic cells (Takahashi et al., “Induction of Pluripotent Stem Cells From Adult Human Fibroblasts By Defined Factors,” Cell 131(5)861-872 (2007); Park et al., “Reprogramming of Human Somatic Cells to Pluripotency With Defined Factors,” Nature (2007); and Yu et al., “Induced Pluripotent Stem Cell Lines Derived From Human Somatic Cells,” Science 318(5858)1917-1920 (2007), each of which is incorporated by reference in its entirety). iPSCs are capable of self-renewal and differentiation into cell fate-committed stem cells, including various types of mature cells. iPSCs exhibit normal morphological (e.g., round shape, large nucleoli, and scant cytoplasm) and growth properties, and express pluripotent cell-specific markers (e.g., Oct-4, SSEA-3, SSEA-4, Tra-1-60, Tra-1-81, but not SSEA-I). iPSCs are substantially genetically identical to their respective differentiated somatic cells of origin, yet display characteristics similar to higher potency cells, such as ES cells. iPSCs can be obtained from various differentiated (e.g., non-pluripotent and multipotent) somatic cells. Although various somatic cells are suitable for iPSC induction, higher reprogramming frequencies are observed when the starting somatic cells have a doubling time of approximately twenty-four hours. Somatic cells useful for carrying out the methods of the present technology include non-embryonic cells obtained from fetal, newborn, juvenile, or adult primates. Preferably, the somatic cells are human somatic cells. Examples of somatic cells include, but are not limited to, bone marrow cells, epithelial cells, fibroblast cells, hematopoietic cells, hepatic cells, intestinal cells, mesenchymal cells, myeloid precursor cells, and spleen cells. Other somatic cells suitable for use in the present technology include CD29+CD44+CD166+CD105+CD73+ and CD31+ mesenchymal cells that attach to a substrate. Alternatively, the somatic cells can be cells that themselves proliferate and differentiate into other types of cells, including blood stem cells, muscle/bone stem cells, brain stem cells, and liver stem cells. Multipotent hematopoietic cells, including myeloid precursor or mesenchymal cells, are also suitable for use in the methods of the technology. Methods for producing and culturing populations of iPSCs are described in WO2008/118820 to Thomson and Yu and WO2007/069666 to Yamanaka, which are hereby incorporated by reference in their entirety.

The term “propagate” means to grow or alter the phenotype of a cell or population of cells. The term “growing” or “expanding” refers to the proliferation of cells in the presence of supporting media, nutrients, growth factors, support cells, or any chemical or biological compound necessary for obtaining the desired number of cells or cell type. In one embodiment, the growing of cells results in the regeneration of tissue. In yet another embodiment, the tissue is comprised of cardiomyocytes.

As used herein, the term “culturing” refers to the in vitro propagation of cells or organisms on or in media of various kinds. It is understood that the descendants of a cell grown in culture may not be completely identical (e.g., morphologically, genetically, or phenotypically) to the parent cell. By “expanded” is meant any proliferation or division of cells. “Clonal proliferation” refers to the growth of a population of cells by the continuous division of single cells into two identical daughter cells and/or population of identical cells.

As used herein, the “lineage” of a cell defines the heredity of the cell, e.g., its predecessors and progeny. The lineage of a cell places the cell within a hereditary scheme of development and differentiation.

As used herein, the term “differentiation” describes the process whereby an unspecialized cell acquires the features of a specialized cell such as a heart, liver, or muscle cell. “Directed differentiation” refers to the manipulation of stem cell culture conditions to induce differentiation into a particular cell type or phenotype. “Dedifferentiated” defines a cell that reverts to a less committed position within the lineage of a cell. As used herein, the term “differentiates or differentiated” defines a cell that takes on a more committed (“differentiated”) position within the lineage of a cell.

As used herein, “a cell that differentiates into a mesodermal (or ectodermal or endodermal) lineage” defines a cell that becomes committed to a specific mesodermal, ectodermal, or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal. Examples of cells that differentiate into ectodermal lineage include, but are not limited to epidermal cells, neurogenic cells, and neurogliagenic cells. Examples of cells that differentiate into endodermal lineage include, but are not limited to pleurogenic cells, and hepatogenic cells, cell that give rise to the lining of the intestine, and cells that give rise to pancreogenic and splanchogenic cells.

As used herein, a “pluripotent cell” defines a less differentiated cell that can give rise to at least two distinct (genotypically and/or phenotypically) further differentiated progeny cells.

A “cardiomyocyte” or “cardiac myocyte” is a specialized muscle cell that primarily forms the myocardium of the heart. Cardiomyocytes have five major components: 1) cell membrane (sarcolemma) and T-tubules, for impulse conduction; 2) sarcoplasmic reticulum, a calcium reservoir needed for contraction; 3) contractile elements; 4) mitochondria; and 5) a nucleus. Cardiomyocytes can be subdivided into subtypes including, but not limited to, atrial cardiomyocyte, ventricular cardiomyocyte, SA nodal cardiomyocyte, peripheral SA nodal cardiomyocyte, or central SA nodal cardiomyocyte. Stem cells can be propagated to mimic the physiological functions of cardiomyocytes or alternatively, differentiate into cardiomyocytes. This differentiation can be detected by the use of markers selected from, but not limited to, myosin heavy chain, myosin light chain, actinin, troponin, and tropomyosin.

The cardiomyocyte marker “myosin heavy chain” and “myosin light chain” are part of a large family of motor proteins found in muscle cells responsible for producing contractile force. These proteins have been sequenced and characterized; see, e.g., GenBank Accession Nos. AAD29948, CAC70714, CAC70712, CAA29119, P12883, NP_000248, P13533, CAA37068, ABR18779, AAA59895, AAA59891, AAA59855, AAB91993, AAH31006, NP_000423, and ABC84220, each of which is incorporated herein by reference. The genes for these proteins have also been sequenced and characterized; see, e.g., GenBank Accession Nos. NM_002472 and NM_000432, each of which is incorporated herein by reference.

The cardiomyocyte marker “actinin” is a microfilament protein which are the thinnest filaments of the cytoskeleton found in the cytoplasm of all eukaryotic cells. Actin polymers also play a role in actomyosin-driven contractile processes and serve as platforms for myosin's ATP hydrolysis-dependent pulling action in muscle contraction. This protein has been sequenced and characterized; see, e.g., GenBank Accession Nos. NP_001093, NP_001095, NP_001094, NP_004915, P35609, NP_598917, NP_112267, AAI07534, and NP_001029807, each of which is incorporated herein by reference. The gene for this protein has also been sequenced and characterized; see, e.g., GenBank Accession Nos. NM_001102, NM_004924, and NM_001103, each of which is incorporated herein by reference.

The cardiomyocyte marker “troponin” is a complex of three proteins that is integral to muscle contraction in skeletal and cardiac muscle. Troponin is attached to the protein “tropomyosin” and lies within the groove between actin filaments in muscle tissue. Tropomyosin can be used as a cardiomyocyte marker. These proteins have been sequenced and characterized; see, e.g., GenBank Accession Nos. NP_000354, NP_003272, P19429, NP_001001430, AAB59509, AAA36771, and NP_001018007, each of which is incorporated herein by reference. The gene for this protein has also been sequenced and characterized; see, e.g., GenBank Accession Nos. NM_000363, NM_152263, and NM_001018007, each of which is incorporated herein by reference.

As used herein, the term “functionally mature” refers to cardiomyoctes (e.g., SC-CM as described herein) that exhibit one or more properties of primary cardiomyocytes (e.g., electrophysiological properties described herein). In some embodiments, “functionally mature cardiomyocytes” are also referred to as “electrophysiologically mature cardiomyocytes” or “physiologically mature cardiomyocytes”.

“Substantially homogeneous” describes a population of cells in which more than approximately 50%, or alternatively more than approximately 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker, e.g. myosin or actin or the expression of a gene or protein.

“Substantially homologous” refers to a nucleic acid or amino acid sequence that is at least approximately 50%, or alternatively more than approximately 60%, or alternatively more than 70%, or alternatively more than 75%, or alternatively more than 80%, or alternatively more than 85%, or alternatively more than 90%, or alternatively, more than 95%, or alternatively more than 99% identical to another nucleic acid sequence or amino acid sequence.

An “effective amount” is an amount sufficient to effect beneficial or desired results. An effective amount can be administered in one or more administrations, applications or dosages.

A “subject,” “individual” or “patient” is used interchangeably herein, and refers to a vertebrate, preferably a mammal, more preferably a human. Mammals include, but are not limited to, murines, rats, simians, bovines, canines, humans, farm animals, sport animals, and pets.

Unmodified cells are sometimes referred to as “source cells” or “source stem cells”. The cells may be prokaryotic or eukaryotic, and include but are not limited to bacterial cells, yeast cells, plant cells, insect cells, animal cells, and mammalian cells, e.g., murines, rats, simians, bovines, canines, porcines, and humans.

In one embodiment, an “immature cell” refers to a cell which does not possess the desired phenotype or genotype. For example, in one embodiment, a mature cell is a cell that is being replaced. The immature cell can be subjected to techniques including physical, biological, or chemical processes which changes, initiates a change, or alters the phenotype or genotype of the cell into a “mature cell.” A “mature cell” refers to a cell which possesses the desired phenotype or genotype. In one embodiment, a mature cell has the phenotype or genotype of, but is not limited to, an adult cardiomyocyte.

### DESCRIPTION

In conventional cultured myocytes, I_{K1} is typically down-regulated and in SC-CM the I_{K1} is either absent or in such low density that resting membrane potentials are volatile (4, 5). This electrophysiological behavior has important implications with respect to the AP, the balance of ionic currents, and the response of the cells to drugs that affect the AP. Without normal membrane polarization, the cardiac sodium channel remains inactivated. Thus, the cellular depolarization response is reduced with low dV/dt and diminished contribution of late sodium current (7, 25). Perhaps more importantly, with a lack of I_{K1}, previous technologies have relied excessively on I_{Kr}, carried by hERG channels, for repolarization (6). The exaggerated role of I_{Kr} in repolarization affects the cellular response to drugs, undermining the utility of SC-CM in drug safety testing. In contrast, embodiments of the I_{K1}-induced SC-CMs have a dV/dt that parallels human ventricular myocytes (e.g., in vivo) and is greater than 4 times higher than the dV/dt of conventional SC-CM. Additionally, repolarization and normal spike-and-dome AP morphology with a stable plateau phase in embodiments of the SC-CM described herein are similar to human cardiomyocytes (e.g., in vivo), and the normal physiologic ionic current balance.

The technology improves arrhythmia modeling and drug safety testing. In particular, the cardiac AP normally exhibits rate adaptation. Accordingly, the same AP frequency should be used to compare AP characteristics between cells or groups of cells tested under different conditions (26), e.g., to compare drug effects on cardiac repolarization at different frequencies. For example, most drug-induced TdP initiates with a short-long-short cycle or is pause dependent (1). As indicated by data collected during the development of embodiments of the technology described herein, I_{K1} mediated normal membrane polarization creates quiescent cells and the AP from these cells can be paced at different frequencies to model bradycardic, tachycardic, or pause dependent arrhythmias and to perform comparisons across experimental platforms.

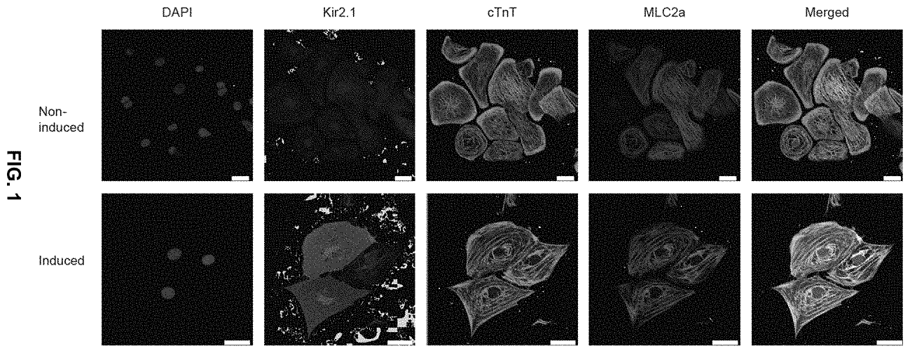

Furthermore, normal membrane polarization by I_{K1} triggers cellular maturation pathways in cardiomyocytes, in skeletal myocytes (e.g., via myogenic transcription factors and myocyte enhancer factor-2), and in other cell types such as osteoblasts (13, 21-23). While it was previously known in the art that contractile proteins decreased in in SC-CMs infected with Kir2.1, experiments conducted during the development of embodiments of the technology described herein indicated that contractile proteins are robustly expressed in the I_{K1}-inducible SC-CM described herein (FIG. 1) (9). Consequently, the presence of I_{K1}-related cellular maturation provides both membrane stabilization and promotes maturation of cardiomyocytes into mature forms that are similar to cells in vivo. Thus, embodiments of the technology provide an improvement over conventional methods for injecting current into cardiomyocytes (12, 24).

In addition, EADs are the triggering mechanism for TdP in LQTS and di-LQTS. During the development of embodiments of the technology described herein, experiments were conducted to analyze EADs from I_{K1}-induced SC-CMs following treatment with drugs that prolong QT (e.g., E4031 and ATX-II). In embodiments of the SC-CMs described herein, E4031 and ATX-II prolonged APD without an effect on depolarization or dV/dt. Further, the slope of the EAD regression analysis is steeply negative, consistent with prior studies on isolated cardiomyocytes from sheep and dogs (8). Importantly, the data indicated that the same or similar E4031 concentration produces AP prolongation and EADs in the I_{K1}-induced SC-CMs described herein as in canine mature myocytes (27).

The technology provides embodiments of I_{K1}-inducible SC-CMs that provide a model system that is electrically comparable to adult human cardiomyocytes. During the development of embodiments of the technology described herein, experiments indicated that the SC-CMs provide a system for modeling cardiac toxicity associated with di-LQTS having AP prolongation and EAD characteristics while maintaining physiologic RMP and excitability.

Accordingly, the technology described herein finds use for modeling arrhythmia and in pre-clinical drug safety testing. Embodiments of the SC-CMs described herein demonstrate an electrically mature phenotype and are amenable to long-term culture; accordingly, embodiments of the SC-CMs described herein easily adapt not only to single cell recordings (e.g., as described in the examples), but also are appropriate for multi-electrode array experiments, 3D constructs, and experiments incorporating a mixture of cell types including fibroblasts to evaluate arrhythmia susceptibility (7, 28).

Compositions

In some embodiments, the technology relates to compositions. In some embodiments, the technology provides a nucleic acid (e.g., a cDNA, a vector, an mRNA) encoding a potassium inward rectifier channel, e.g., a Kir. In some embodiments, the technology provides a nucleic acid (e.g., a cDNA, a vector, an mRNA) encoding a Kir1, Kir2, Kir3, Kir4, Kir5, Kir6, or Kir7 potassium inward rectifier channel. In some embodiments, the technology provides a nucleic acid (e.g., a cDNA, a vector, an mRNA) encoding a Kir2 subclass potassium inward rectifier channel (e.g., Kir2.1, Kir2.2, Kir2.3, or Kir2.4). In some embodiments, the technology provides a nucleic acid comprising a sequence encoding a Kir2.1 potassium inward rectifier channel. In some embodiments, the technology provides a nucleic acid comprising a nucleotide sequence as provided by SEQ ID NO: 1. In some embodiments, the technology provides a nucleotide sequence encoding a protein having an amino acid sequence as provided by SEQ ID NO: 2.

In some embodiments, the technology provides a nucleic acid encoding a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel in frame with (e.g., translationally linked to) a tag such as, e.g., a green fluorescent protein.

In some embodiments, the technology provides a nucleic acid comprising one or more mutations, e.g., encoding a substituted variant of a potassium inward rectifier channel, e.g., a Kir. In some embodiments, the technology provides a nucleic acid comprising one or more mutations (e.g., a cDNA, a vector, an mRNA comprising one or more mutations), e.g., encoding a substituted variant of a Kir1, Kir2, Kir3, Kir4, Kir5, Kir6, or Kir7 potassium inward rectifier channel. In some embodiments, the technology provides a nucleic acid comprising one or more mutations (e.g., a cDNA, a vector, an mRNA comprising one or more mutations), e.g., encoding a substituted variant of a Kir2 subclass potassium inward rectifier channel (e.g., Kir2.1, Kir2.2, Kir2.3, or Kir2.4). In some embodiments, the technology provides a nucleic acid comprising a sequence comprising one or more mutations, e.g., encoding a substituted variant of a Kir2.1 potassium inward rectifier channel. In some embodiments, the technology provides a nucleic acid comprising a nucleotide sequence having 75, 80, 85, 90, 91, 92, 93, 94, 95, 96, 97, 98, 99, 99.5, or 99.9% identity to the nucleotide sequence provided by SEQ ID NO: 1. In some embodiments, the technology provides a nucleotide sequence encoding a protein having an amino acid sequence having 75, 80, 85, 90, 91, 92, 93, 94, 95, 96, 97, 98, 99, 99.5, or 99.9% identity to the amino acid sequence provided by SEQ ID NO: 2.

In some embodiments, the technology provides a vector, plasmid, or other construct comprising a nucleic acid encoding a potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel. In some embodiments, the technology provides a host (e.g., a prokaryotic, archaeal, and/or eukaryotic host) comprising a nucleic acid encoding a potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))). In some embodiments, the technology provides a host (e.g., a prokaryotic, archaeal, and/or eukaryotic host) comprising a vector, plasmid, or other construct comprising a nucleic acid encoding a potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel.

Experiments conducted during the development of embodiments of the technology described herein indicated that cardiomyocyte myofibril production and organization were affected by the presence of Kir2.1. In particular, cultured stem cells that were infected with Kir2.1 adenovirus did not produce normal myofibrils with normal structural organization unless the cells were externally paced. However, pacing causes cellular stress in early cell differentiation (e.g., in cells less than approximately 30 days old) and is therefore not appropriate for all methods of producing differentiated cells. Thus, an inducible promoter is used in some embodiments to produce appropriately developed (e.g., differentiated) cells as described herein. Furthermore, use of an inducible promoter provides a technology with improved usefulness and efficiencies. In addition, in some embodiments, the technology comprises use of a Kir2.1 cloned into other cell types (e.g., non-cardiomyocytes (e.g., neurocytes)) and the inducible promoter provides a technology for inducing the expression of Kir2.1 in other cell types.

Accordingly, in some embodiments, the technology provides a nucleic acid encoding a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel under the control of an inducible promoter. In some embodiments, the technology provides a nucleic acid encoding a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel under the control of a promoter that is inducible by doxycycline (e.g., a TRE3G (tTA-activated) promoter).

The technology is not limited to the doxycycline-inducible promoter and contemplates the use of other promoters (e.g., inducible and constitutive promoters) compatible for use in eukaryotic cells (e.g., cells comprising Kir2.1 (e.g., cardiomyocytes)). For example, in some embodiments, constructs comprise the constitutive myosin heavy chain (MHC) promoter. In some embodiments, constructs comprise a promoter appropriate for constitutive or induced expression in the cell type in which the construct will be expressed. For example, in some embodiments, a construct comprising a cloned Kir2.1 for introduction into a neurocyte comprises a promoter activated and/or inducible in a neurocyte. In some embodiments, the technology provides a nucleic acid encoding a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel under the control of a constitutive promoter (e.g., the constitutive myosin heavy chain (MHC) promoter).

In some embodiments, the technology provides a plasmid comprising: 1) a nucleic acid encoding a transcriptional activator (e.g., a tetracycline transcriptional activator (e.g., a tTA)) under the control of a promoter (e.g., a constitutive promoter (e.g., the CAG promoter)); and 2) a nucleic acid encoding a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel under the control of a promoter induced by the transcriptional activator (e.g., a tetracycline transcriptional activator (e.g., a tTA)). The CAG promoter is described, e.g., in Alexopoulou et al. (2008) “The CMV early enhancer/chicken beta actin (CAG) promoter can be used to drive transgene expression during the differentiation of murine embryonic stem cells into vascular progenitors” BMC Cell Biology 9: 2; Miyazaki et al. (1989). “Expression vector system based on the chicken beta-actin promoter directs efficient production of interleukin-5” Gene 79: 269-77; and Niwa et al. (1991) “Efficient selection for high-expression transfectants with a novel eukaryotic vector” Gene 108: 193-9, each of which is incorporated herein by reference.

Further embodiments provide a cell comprising an inducible Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel. In some embodiments, the technology provides a cell with an inducible I_{K1} current. In some embodiments, the cell is a stem cell (e.g., a differentiated stem cell). In some embodiments, the stem cell is an embryonic stem cell or a pluripotent stem cell. In some embodiments, the cell is an induced pluripotent stem cell. In some embodiments, the differentiated stem cell is a stem-cell derived cell such as, e.g., a muscle cell (e.g., a heart cell (e.g., a cardiomyocyte)), a neurocyte, an endocrine cell, or other cell that has action potentials and/or that is an electrically excitable cell. Thus, in some embodiments the technology provides a cell (e.g., a stem cell, a stem-cell derived cell (e.g., a muscle cell (e.g., a heart cell (e.g., a cardiomyocyte))) comprising an inducible Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel. In some embodiments, the technology provides a cell (e.g., a stem cell, a stem-cell derived cell (e.g., a muscle cell (e.g., a heart cell (e.g., a cardiomyocyte))) comprising an inducible I_{K1} current. In some embodiments, the cell is derived from a stem cell line that is an embryonic stem cell line. In some embodiments, the cell is derived from a stem cell line that is a WA09 (H9) stem cell line. In some embodiments the cell is derived from a stem cell line that is an induced pluripotent stem cell line. In some embodiments, the cell is derived from a stem cell line that is a 19-9-11 stem cell line. In some embodiments, the cell is derived from a cell line previously established to differentiate efficiently to the desired cell type. For example, in embodiments in which a cardiomyocyte comprises the inducible Kir, the cell is derived from a cell line previously established to differentiate efficiently to a cardiomyocyte.

As discussed herein, stem-cell derived cardiomyocytes expressing Kir2.1 become electrically mature and closely simulate the biological (e.g., physiological (e.g., electrophysiological)) response of cardiomyocytes in vivo. Furthermore, stem-cell derived cardiomyocytes expressing Kir2.1 proceed on a maturation pathway that parallels the maturation pathway in vivo. For instance, stem-cell derived cardiomyocytes expressing Kir2.1 exhibit increased myofibril production, bi-nucleation was more common, and the cells were larger.

In some embodiments, the technology comprises a stem-cell derived cardiomyocyte a (e.g., a ventricular cell or ventricular-like cell) comprising an inducible Kir2.1. In some embodiments, the technology comprises a stem-cell derived cardiomyocyte a (e.g., an atrial cell or atrial-like cell) comprising an inducible Kir2.1 and/or Kir2.3.

In addition, pacing cardiomyocytes improves myofibril development in vitro.

Accordingly, in some embodiments, the technology further comprises electrically pacing embodiments of cells described herein. In some embodiments, the technology comprises inducing the expression of a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel in a cell (e.g., a stem cell, a stem-cell derived cell (e.g., a muscle cell (e.g., a heart cell (e.g., a cardiomyocyte))) comprising an inducible Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel and applying electrical pacing to said cell. In some embodiments, the technology comprises inducing a I_{K1} current in a cell (e.g., a stem cell, a stem-cell derived cell (e.g., a muscle cell (e.g., a heart cell (e.g., a cardiomyocyte))) and applying electrical pacing to said cell. As used herein, the term “functionally mature cardiomyocytes” refers to cardiomyoctes that exhibit one or more properties of primary cardiomyocytes (e.g., electrophysiological properties described herein). In some embodiments, “functionally mature cardiomyocytes” are also referred to as “physiologically mature cardiomyocytes” or “electrophysiologically mature cardiomyocytes.”

Cells

The technology is not limited in the cells. Accordingly, in various embodiments many types of cells (e.g., cultured cells, stem cells, synthetic cells) are employed with the technology described herein. In some embodiments, the cell is a pluripotent cell with potential for cardiomyocyte differentiation. Such cells include embryonic stem cells and induced pluripotent stem cells, regardless of source. For example, induced pluripotent stem cells may be derived from stem cells or adult somatic cells that have undergone a dedifferentiation process. Exemplary cells that are included in the scope of embodiments of the technology include muscle cells, cardiomyocytes, neurons, stem cell-derived cardiomyocytes, stem cell-derived neurons, cells comprising ion channels, cells comprising a proton pump, etc.

Induced pluripotent stem cells (iPSCs) may be generated using any known approach. In some embodiments, iPSCs are obtained from adult human cells (e.g., fibroblasts). In some embodiments, modification of transcription factors (e.g., Oct3/4, Sox family members (Sox2, Sox1, Sox3, Sox15, Sox18), Klf Family members (Klf4, Klf2, Klf1, Klf5), Myc family members (c-myc, n-myc, l-myc), Nanog, LIN28, Glis1, etc.), or mimicking their activities is employed to generate iPSCs (e.g., using a transgenic vector (adenovirus, lentivirus, plasmids, transposons, etc.), inhibitors, delivery of proteins, microRNAs, etc.).

In some embodiments, the cells are stem cell-derived cardiomyocytes. In some embodiments, cells are modified to include a marker and used as diagnostic compositions to assess properties of the cells in response to changes in their environment.

In some embodiments, the cells are stem cell-derived cardiomyocytes that are ventricular SC-CMs or ventricular-like SC-CMs. While the technology is not limited to ventricular SC-CMs or ventricular-like SC-CMs, embodiments of the technology described herein were developed during experiments using ventricular SC-CMs or ventricular-like SC-CMs because torsades and other fatal arrhythmias are generated from the ventricle, not the atrium. Accordingly, embodiments of the technology related to modeling ventricular arrhythmia susceptibility comprise use of ventricular SC-CMs or ventricular-like SC-CMs. However, the technology is not limited to ventricular SC-CMs or ventricular-like SC-CMs and thus includes other stem cell-derived cardiomyocytes such as atrial SC-CMs, atrial-like SC-CMs, and other cardiomyocytes.

Methods

In some embodiments, the technology provides methods for constructing SC-CMs comprising an inducible potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel. Embodiments of the technology relate to methods for preparing SC-CMs comprising an inducible Kir2.1. Some embodiments of the technology relate to methods for preparing SC-CMs comprising an inducible I_{K1} current. Additional embodiments relate to methods of using SC-CMs comprising an inducible Kir2.1 and/or methods of using SC-CMs comprising an inducible I_{K1} current. In some embodiments, the technology finds use in testing the cardiac safety of drugs, for modeling cardiac abnormalities (e.g., arrhythmias, long QT (e.g., drug-induced long QT syndrome), AP anomalies (e.g., AP prolongation), early afterdepolarizations, etc.), and studying physiologically relevant characteristics of cardiac cells.

Embodiments for preparing SC-CMs comprising an inducible Kir2.1 and/or inducible I_{K1} current comprise steps of, e.g., providing a stem cell (e.g., an embryonic stem cell, an induced pluripotent stem cell, or other cell described herein). Embodiments comprise cloning a potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel and/or obtaining or providing a nucleic acid (e.g., a cloned nucleic acid) encoding a potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel. In some embodiments, the potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel and/or obtaining or providing a nucleic acid (e.g., a cloned nucleic acid) encoding a potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel is operably linked to an inducible promoter (e.g., a doxycycline-inducible promoter (e.g., a TRE3G (tTA-activated) promoter)). In some embodiments, the potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel and/or obtaining or providing a nucleic acid (e.g., a cloned nucleic acid) encoding a potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel is operably linked to constitutive promoter.

Further, embodiments comprise introducing a cloned potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel into a stem cell (e.g., an embryonic stem cell, an induced pluripotent stem cell, or other cell described herein). In some embodiments, introducing a cloned potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel into a stem cell (e.g., an embryonic stem cell, an induced pluripotent stem cell, or other cell described herein) comprises use of a CRISPR technology (e.g., a Cas9 or Cas9-like protein (e.g., a CRISPR nickase)) and one or more gRNAs targeting a chromosomal site at which the cloned potassium inward rectifier channel will be introduced (e.g., integrated). In some embodiments, the present technology comprises providing and/or using a Cas9 protein from S. pyogenes, either as encoded in bacteria or codon-optimized for expression in mammalian cells. In some embodiments, a Cas9 polypeptide comprises a mutation (e.g., an amino acid substitution) that produces a Cas9 enzyme having a “nickase” activity. In some embodiments, the Cas9 nickase has a substitution at the aspartic acid at position 10, the glutamic acid at position 762, the histidine at position 983, or the aspartic acid at position 986 (e.g., at D10, E762, H983, or D986). Substitutions at these positions are, in some embodiments, alanine (e.g., a D10A, E762A, H983A, or D986A substitution); see, e.g., Nishimasu (2014) Cell 156: 935-949, incorporated herein by reference). In particular, embodiments use a Cas9 mutant (e.g., D10A) that cuts only one strand of DNA (e.g., a Cas9 nickase), thus generating single-strand breaks (SSB), e.g., that are faithfully repaired without inducing indels. The nickase activity of the mutant Cas9 nickase proteins is in contrast to wild-type Cas9 proteins that generate blunt double-strand breaks. Thus, in some embodiments, the sequence of a S. pyogenes dCas9 protein having a substitution of alanine for asp artic acid at position 10 finds use in the technology provided herein, e.g., as described in Nishimasu (2014) Cell 156: 935-949, incorporated herein by reference.

Accordingly, embodiments comprise a step of designing, obtaining, providing, and/or synthesizing a guide RNA targeting the chromosomal site at which the cloned potassium inward rectifier channel will be introduced (e.g., integrated). In some embodiments, the CRISPR system (e.g., the gRNA) targets the AAVS1 locus. In some embodiments, the technology comprises a step of electroporating to introduce one or more nucleic acids (e.g., a gRNA and/or a cloned potassium inward rectifier channel) into a cell.

Embodiments comprise a step of culturing the stem cells before and/or after introducing the cloned potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel into a stem cell.

In some embodiments, the methods comprise analyzing SC-CMs comprising an inducible potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel, e.g., to confirm the clones and/or to confirm the absence of off-target effects. In some embodiments, analysis comprises use of amplification. In some embodiments, analysis comprises identifying a clone having a normal karyotype, a normal genotype, and/or no off-target effects. In some embodiments, analyzing SC-CMs comprising an inducible potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel comprises use of microscopy, immunocytochemistry, immunohistochemistry, and/or cellular electrophysiology.

For example, in some embodiments cells are stained (e.g., using dyes and/or detectable antibodies) to assess the presence and/or absence of certain molecular markers associated with differentiated stem cells, undifferentiated stem cells, a potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel, an inducible potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel, or proper karyotype. Some embodiments relate to detecting one or more markers associated with a mature cardiomyocyte phenotype, e.g., detecting MLC2v and/or Troponin I. In some embodiments, markers are detected by immunostaining and/or by flow cytometry.

Some embodiments comprise use of electrophysiological measurements to measure cellular currents, e.g., associated with one or more ion channels. For instance, some embodiments comprise recording an I_{K1} current, an I_{f} current, and/or currents associated with other small ions transferring across a cell membrane (e.g., H+, K+, Na+, Ca++, electrons, etc.) Some embodiments comprise recording an AP; AP amplitude; resting membrane potential; AP duration at 10% (APD10), 50% (APD50), 70% (APD70), and 90% (APD90) of repolarization; maximum upstroke velocity (dV/dtmax), etc.

Some embodiments comprise a step of differentiating a stem cell (e.g., an undifferentiated stem cell) comprising an inducible potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel, e.g., to prepare SC-CMs comprising an inducible potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel. Some embodiments comprise storing (e.g., by freezing (e.g., in liquid nitrogen)) SC-CMs comprising an inducible potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel.

Some embodiments comprise inducing the I_{K1} current and/or inducing the expression of the potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel. For instance, in embodiments related to a potassium inward rectifier channel (e.g., inducible Kir2.1) under the control of a doxycycline-induced promoter, the methods comprise adding doxycycline to a cell (e.g., a cell culture and/or cell suspension), contacting a cell with doxycycline, and/or otherwise providing conditions such that doxycycline accesses the interior of a cell, contacts a tetracycline transcriptional transactivator (tTA), and/or effects the translocation of the tTA protein from the cytoplasm to the nucleus where it activates a TRE3G (tTA-activated) promoter operably linked to the cloned potassium inward rectifier channel, where it subsequently drives expression of the potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel. The technology is not limited to the doxycycline/tTA/TRE3G inducible system and encompasses methods of constructing and inducing cloned nucleic acids under the control of inducible expression systems.

Some embodiments of methods relate to using SC-CMs comprising an inducible Kir2.1 and/or methods of using SC-CMs comprising an inducible I_{K1} current. In some embodiments, the technology finds use in testing the cardiac safety of drugs, for modeling cardiac abnormalities (e.g., arrhythmias, long QT (e.g., drug-induced long QT syndrome), AP anomalies (e.g., AP prolongation), early afterdepolarizations, etc.), and studying physiologically relevant characteristics of cardiac cells.

For instance, in some embodiments, SC-CMs comprising an inducible potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel are used to screen for factors (such as solvents, small molecule drugs, peptides, oligonucleotides) or environmental conditions (such as culture conditions or manipulation) that affect the characteristics of such cells and their various progeny.

In some embodiments, SC-CMs comprising an inducible potassium inward rectifier channel are induced to express the potassium inward rectifier channel (e.g., a Kir2.1) and grown to provide SC-CMs comprising a physiological mature cardiomyocyte phenotype. In some embodiments, SC-CMs comprising an inducible potassium inward rectifier channel are induced to express the potassium inward rectifier channel (e.g., a Kir2.1), paced by providing an external current, and grown to provide SC-CMs comprising a physiological mature cardiomyocyte phenotype. Embodiments of methods comprise use of these physiologically mature SC-CMs.

Accordingly, related methods comprise contacting physiologically mature SC-CMs with compositions (such as solvents, small molecule drugs, peptides, oligonucleotides) or exposing physiologically mature SC-CMs to environmental conditions (such as culture conditions or manipulation) that affect the characteristics of these cells.

In some applications, physiologically mature SC-CMs are used to test pharmaceutical compounds for their effect on cardiac muscle tissue maintenance or repair. Screening may be done either because the compound is designed to have a pharmacological effect on cardiac cells, or because a compound designed to have effects elsewhere may have unintended side effects on cardiac cells.

Accordingly, embodiments provide contacting physiological mature SC-CMs with a pharmaceutical compound, e.g., to assess the effect of the pharmaceutical compound on cardiac muscle tissue maintenance or repair. Assessment of the activity of candidate pharmaceutical compounds generally involves combining the physiologically mature SC-CMs with the candidate compound, either alone or in combination with other drugs. The investigator determines any change in the morphology, marker phenotype, or functional activity of the cells that is attributable to the compound (compared with untreated cells or cells treated with an inert compound), and then correlates the effect of the compound with the observed change. Thus, embodiments of methods comprise measuring the morphology, marker phenotype, or functional activity (e.g., electrophysiological characteristics) of the cells in the presence and/or absence of one or more pharmaceutical compound.

In some embodiments, cytotoxicity is determined by the effect of a compound on cell viability, survival, morphology, and/or the expression of certain markers and receptors of the physiologically mature SC-CMs. For instance, effects of a drug on chromosomal DNA can be determined by measuring DNA synthesis or repair (e.g., using [3H]-thymidine or BrdU incorporation). Unwanted effects can also include unusual rates of sister chromatid exchange, determined by metaphase spread. See, e.g., A. Vickers (pp 375-410 in In vitro Methods in Pharmaceutical Research, Academic Press, 1997).

In some embodiments, the effect of a composition and/or environmental condition on the cell function of the SC-CMs disclosed herein (e.g., physiologically mature SC-CMs) is assessed using any standard assay to observe phenotype or activity of SC-CMs, such as marker expression, receptor binding, contractile activity, or electrophysiology. Pharmaceutical candidates can also be tested for their effect on contractile activity—such as whether they increase or decrease the extent or frequency of contraction. Where an effect is observed, the concentration of the compound can be titrated to determine the median effective dose (ED50). See, e.g., In vitro Methods in Pharmaceutical Research, Academic Press, 1997, and U.S. Pat. No. 5,030,015, each of which is incorporated herein by reference.

Systems

Some embodiments relate to systems comprising SC-CMs comprising an inducible potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel in an induced or non-induced state. In some embodiments, systems comprise an inducer (e.g., doxycycline), e.g., to provide SC-CMs comprising an inducible potassium inward rectifier channel in an induced state. In some embodiments, systems comprise a component to pace cells (e.g., to provide an appropriate current to induce pacing in cardiomyocytes), e.g., comprising one or more electrodes, wires, current source, etc. In some embodiments, systems comprise a dye (e.g., to detect a biomarker) and a component to detect the dye (e.g., a fluorescence microscope, a flow cytometer, etc.)

In some embodiments, systems comprise a component to measure one or more electrophysiological characteristics of cardiomyocytes, e.g., a voltage clamp, current clamp, patch-clamp, or sharp electrode, planar patch clamp, a Bioelectric Recognition Assay (BERA) component, and/or exemplary system components as described in, e.g., U.S. Pat. Nos. 7,270,730; 5,993,778; and 6,461,860, and that are described in Hamill et al. (1981) Pflugers Arch. 391(2)85-100; Alvarez et al. (2002) Adv. Physiol. Educ. 26(1-4)327-341; Kornreich (2007) J. Vet. Cardiol. 9(1)25-37; Perkins (2006) J. Neurosci. Methods. 154(1-2)1-18; Gurney (2000) J. Pharmacol. Toxicol. Methods. 44(22)309-420; Baker et al. (1999) J. Neurosci. Methods 94(1)5-17; McNames and Pearson (2006) Conf. Proc. IEEE Eng. Med. Biol. Soc. 1(1): 1185-1188; Porterfield (2007) Biosens. Bioelectron. 22(7)1186-1196; Wang and Li (2003) Assay Drug Dev. Technol. 1(5)695-708; and Kintzios et al. (2001) Biosens. Bioelectron. 16(4-5)325-336, each of which is included herein by reference. In some embodiments, systems comprise software, e.g., to collect, analyze, and present data, and a microcontroller (e.g., computer) to implement embodiments of methods for collecting, analyzing, and presenting data.

Although the disclosure herein refers to certain illustrated embodiments, it is to be understood that these embodiments are presented by way of example and not by way of limitation.

### EXAMPLES

Materials and Methods

Cloned Kir2.1

Human cDNA was used to clone a cDNA encoding the potassium (K) inward rectifier 2.1 (Kir2.1) into pcDNA3.1. Wild-type (WT) human Kir2.1 was isolated by PCR from human cardiac cDNA using forward primer atgggcagtgtgcgaaccaac (SEQ ID NO: 3) and reverse primer tcatatctccgactctcgccgtaagg (SEQ ID NO: 4) (see, e.g., Reference 15: Eckhardt et al. (2007) “KCNJ2 mutations in arrhythmia patients referred for LQT testing: a mutation T305A with novel effect on rectification properties” Heart Rhythm 4: 323-29, incorporated herein by reference in its entirety). The resulting human KCNJ2 cDNA sequence was verified by sequencing and is provided below (SEQ ID NO: 1):

ATTTTTTTGGTGTGTGTGTCTTCACCGAACATTCAAAACTGTTTCTCCAA
AGCGTTTTGCAAAAACTCAGACTGTTTTCCAAAGCAGAAGCACTGGAGTC
CCCAGCAGAAGCGATGGGCAGTGTGCGAACCAACCGCTACAGCATCGTTT
CTTCAGAAGAAGACGGTATGAAGTTGGCCACCATGGCAGTTGCAAATGGC
TTTGGGAACGGGAAGAGTAAAGTCCACACCCGACAACAGTGCAGGAGCCG
CTTTGTGAAGAAAGATGGCCACTGTAATGTTCAGTTCATCAATGTGGGTG
AGAAGGGGCAACGGTACCTCGCAGACATCTTCACCACGTGTGTGGACATT
CGCTGGCGGTGGATGCTGGTTATTTTCTGCCTGGCTTTCGTCCTGTCATG
GCTGTTTTTTGGCTGTGTGTTTTGGTTGATAGCTCTGCTCCATGGGGACC
TGGATGCATCCAAAGAGGGCAAAGCTTGTGTGTCCGAGGTCAACAGCTTC
ACGGCTGCCTTCCTCTTCTCCATTGAGACCCAGACAACCATAGGCTATGG
TTTCAGATGTGTCACGGATGAATGCCCAATTGCTGTTTTCATGGTGGTGT
TCCAGTCAATCGTGGGCTGCATCATCGATGCTTTCATCATTGGCGCAGTC
ATGGCCAAGATGGCAAAGCCAAAGAAGAGAAACGAGACTCTTGTCTTCAG
TCACAATGCCGTGATTGCCATGAGAGACGGCAAGCTGTGTTTGATGTGGC
GAGTGGGCAATCTTCGGAAAAGCCACTTGGTGGAAGCTCATGTTCGAGCA
CAGCTCCTCAAATCCAGAATTACTTCTGAAGGGGAGTATATCCCTCTGGA
TCAAATAGACATCAATGTTGGGTTTGACAGTGGAATCGATCGTATATTTC
TGGTGTCCCCAATCACTATAGTCCATGAAATAGATGAAGACAGTCCTTTA
TATGATTTGAGTAAACAGGACATTGACAACGCAGACTTTGAAATCGTGGT
CATACTGGAAGGCATGGTGGAAGCCACTGCCATGACGACACAGTGCCGTA
GCTCTTATCTAGCAAATGAAATCCTGTGGGGCCACCGCTATGAGCCTGTG
CTCTTTGAAGAGAAGCACTACTACAAAGTGGACTATTCCAGGTTCCACAA
AACTTACGAAGTCCCCAACACTCCCCTTTGTAGTGCCAGAGACTTAGCAG
AAAAGAAATATATCCTCTCAAATGCAAATTCATTTTGCTATGAAAATGAA
GTTGCCCTCACAAGCAAAGAGGAAGACGACAGTGAAAATGGAGTTCCAGA
AAGCACTAGTACGGACACGCCCCCTGACATAGACCTTCACAACCAGGCAA
GTGTACCTCTAGAGCCCAGGCCCTTACGGCGAGAGTCGGAGATATGACTG
ACTGATTCCTTCTCTGGAATAGTTACTTTACAACACGGTCTGTTGGTCAG
AGGCCCAAAACAGTTATACAGATGACGGTACTGGTCAAGATGGGTCAAGC
AAGCGGCCACACGGGACTGAGGC

The translated amino acid sequence corresponding to the cloned human Kir2.1 nucleotide sequence above is provided below (SEQ ID NO: 2):

MGSVRTNRYSIVSSEEDGMKLATMAVANGFGNGKSKVHTRQQCRSRFVKK
DGHCNVQFINVGEKGQRYLADIFTTCVDIRWRWMLVIFCLAFVLSWLFFG
CVFWLIALLHGDLDASKEGKACVSEVNSFTAAFLFSIETQTTIGYGFRCV
TDECPIAVFMVVFQSIVGCIIDAFIIGAVMAKMAKPKKRNETLVFSHNAV
IAMRDGKLCLMWRVGNLRKSHLVEAHVRAQLLKSRIISEGEYIPLDQIDI
NVGFDSGIDRIFLVSPITIVHEIDEDSPLYDLSKQDIDNADFEIVVILEG
MVEATAMTTQCRSSYLANEILWGHRYEPVLFEEKHYYKVDYSRFHKTYEV
PNTPLCSARDLAEKKYILSNANSFCYENEVALTSKEEDDSENGVPESTST
DTPPDIDLHNQASVPLEPRPLRRESEI

Homology between the human sequence and other species is high. No known differences in regulation or conductance were identified. During the development of embodiments of the technology, experiments were conducted to clone Kir2.1 with a green fluorescent protein (GFP) tag. Accordingly, in some embodiments, the technology comprises use of a GFP-tagged Kir2.1 construct. In some embodiments, the technology comprises use of a non-tagged Kir2.1construct.

Inducible Kir2.1 Construct

During the development of embodiments of the technology provided herein, experiments were conducted in which Kir2.1 was cloned into a vector with an inducible promoter (e.g., a doxycycline-inducible promoter). A human cDNA clone of Kir2.1 was isolated and sequenced as described above (see, e.g., Eckhardt et al. (2007) “KCNJ2 mutations in arrhythmia patients referred for LQT testing: a mutation T305A with novel effect on rectification properties” Heart Rhythm 4: 323-29, incorporated herein by reference in its entirety). A CRISPR donor plasmid was constructed using the Kir2.1 cDNA and a doxycycline-inducible plasmid (see, e.g., Chen et al (2014) “Modeling ALS with iPSCs reveals that mutant SOD1 misregulates neurofilament balance in motor neurons” Cell Stem Cell 14: 796-809; Qian et al. (2014) “A simple and efficient system for regulating gene expression in human pluripotent stem cells and derivatives” Stem Cells 32: 1230-38, each of which is incorporated herein by reference). The donor plasmid comprises homology arms representing approximately 800 bp upstream and downstream of the AAVS1 Cas9-targeted locus to facilitate homologous repair. Between the homology arms, the plasmid comprises the following components: 1) a tetracycline transcriptional transactivator (tTA) protein under the control of the constitutive CAG promoter; and 2) a TRE3G (tTA-activated) promoter driving expression of the cloned KIR2.1 cDNA sequence. When doxycycline is added to cells, the tTA protein translocates from the cytoplasm to the nucleus, where it subsequently drives KIR2.1 expression.

Stem Cell Derived-Cardiomyocytes

H9 cells (WA09) (14) (WiCell, Madison, Wis.), an established stem cell (SC) line, and 19-9-11 cells, an induced pluripotent stem cell line, were modified using CRISPR-Cas9 to produce H9 and 19-9-11 cells comprising an inducible Kir2.1 (“ES-Kir2.1” and “iPS-Kir2.1”).

Different stem cell lines differentiate into various cell types (e.g., cardiomyocytes, neural cells, hepatocytes, etc.) with different efficiencies. Accordingly, experiments conducted during the development of embodiments of the technology described herein used cells previously established to differentiate efficiently to cardiomyocytes. In particular, experiments used the embryonic stem cell line WA09 (H9) and the induced pluripotent stem cell line 19-9-11. However, the technology is not limited to the use of these or other stem cell lines and the technology contemplates the use of any stem cell line that can be differentiated into the desired cell types for the technology.

The cloned Kir2.1 was introduced into H9 cells (16) at the AAVS1 locus using the doxycycline-inducible donor plasmid comprising the cloned Kir2.1 described above and constructs expressing single guide RNA (sgRNA) sequences targeting the AAVS1 locus. Constructs expressing sgRNA sequences that target the AAVS1 locus were cloned into a Cas9 sgRNA plasmid from the laboratory of Su-Chun Zhang (Addgene ID 68463; see, e.g., Chen et al. (2015) “Engineering Human Stem Cell Lines with Inducible Gene Knockout using CRISPR/Cas9” Cell Stem Cell 17: 233-44, which is incorporated herein along with its supplemental data and information in their entireties). During the development of embodiments of the technology described herein, A D10A mutant CRISPR nickase enzyme was used with sgRNA targeting both the positive (+) and negative (−) strands of DNA (e.g., as identified using the crispr.mit.edu site).

Cells were cultured and electroporated as previously described (see, e.g., Chen et al. (2015) “Engineering Human Stem Cell Lines with Inducible Gene Knockout using CRISPR/Cas9” Cell Stem Cell 17: 233-44, which is incorporated herein along with its supplemental data and information in their entireties). Human ESCs or iPSCs were cultured in hPSC medium on mouse embryonic fibroblast (MEF) feeder cells with Rho Kinase (ROCK)-inhibitor (0.5 μM, Calbiochem, H-1152P) for 24 hours prior to electroporation. Cells were digested by TrypLE express Enzyme (Life Technologies) for 3-4 minutes, washed two times with DMEM/F12, and harvested in hPSC medium with 0.5 μM ROCK-inhibitor. Cells were dispersed into single cells and 1×10⁷ cells were electroporated with plasmids (see below) in 500 μl of Electroporation Buffer (KCl 5 mM, MgCl₂ 5 mM, HEPES 15 mM, Na₂HPO₄ 102.94 mM, NaH₂PO₄ 47.06 mM, pH=7.2) using the Gene Pulser Xcell System (Bio-Rad) at 250 V, 500 μF in 0.4-cm cuvettes (Phenix Research Products).

Cells were electroporated in a cocktail of 15 μg of CAG-Cas9D10A plasmid (Kiran Musunuru, Addgene ID 44720), 15 μg each sgRNA plasmid (sgRNA-#1 and sgRNA-#3), and 30 μg of a donor plasmid targeting the AAVS1 genetic locus. Following electroporation, cells were plated on MEF feeders in 0.5 μM ROCK inhibitor and 5 μM L-755507 (beta 3-adrenergic receptor agonist), which biases cells toward homologous repair (e.g., homology-directed repair (HDR)) relative to non-homologous end joining (NHEJ) (See, e.g., Chen et al. (2015) “Small Molecules Enhance CRISPR Genome Editing in Pluripotent Stem Cells” Cell Stem Cell 16: 142-47, incorporated herein by reference in its entirety). At 72 hours post-electroporation, cells were treated with puromycin (0.5 μg/mL, Invivogen, ant-pr-1) to select for cells incorporating the plasmid. Concurrent with puromycin treatment, the cells were fed with MEF-conditioned hPSC media. Puromycin treatment was increased to 1.0 μg/mL on day 16 post-electroporation and maintained at this level until colonies were sufficiently sized for selection. Puromycin was removed and 0.5 μM ROCK inhibitor was added 24 hours prior to clone picking.

Single-cell colonies were manually selected and mechanically disaggregated for genotype analysis to confirm clones with no off-target effects. Analysis of potential off-target genome modification produced by the CRISPR-Cas9 system was performed by identifying the five highest-likelihood off-target sites predicted by the crispr.mit.edu algorithms and designing genotyping primers to amplify these regions. Genomic DNA for genotyping was isolated from colonies using QuickExtract DNA Extraction Solution 1.0 (Epicentre). PCR products were produced for genotyping and sequencing using Q5 polymerase-based PCR (NEB) and the genotyping primers.

Genotyping included identifying positive insertions using the primer pair:

AAVS 5′arm F
SEQ ID NO: 5
CATGCAGTCCTCCTTACCATC
AAVS 5′arm R
SEQ ID NO: 6
AGGAAGAGTTCTTGCAGCTC

Homozygous genotypes were distinguished from heterozygous genotypes using the primer pair:

AAVS 5′arm F
SEQ ID NO: 5
CATGCAGTCCTCCTTACCATC
AAVS1 3′ close seq R
SEQ ID NO: 7
TCCTCTCTGGCTCCATCGTA

PCR using the primers AAVS 5′arm F and AAVS1 3′ close seq R produces a product of approximately 1 Kbp from heterozygous clones and produces no product from homozygous clones.

Off-target insertions were identified using the primer pair:

T3 Forward
SEQ ID NO: 8
GCAATTAACCCTCACTAAAGG
AAVS 5′arm R
SEQ ID NO: 6
AGGAAGAGTTCTTGCAGCTC

PCR using the primers T3 Forward and AAVS 5′arm R produces a product from whole plasmid or insertions that resulted from mechanisms other than homologous recombination (e.g., non-homologous recombination mediated insertions).

The qPCR system was validated using the following primer pair:

KIR2.1 Q-1F
SEQ ID NO: 9
TCCGAGGTCAACAGCTTCAC
KIR2.1 Q-1R
SEQ ID NO: 10
TTGGGCATTCATCCGTGACA

Potential off-target genome modification produced by the CRISPR-Cas9 system and guide RNAs sgRNA-#1 and sgRNA-#3 was evaluated using qPCR and the following primer pairs developed using the crispr.mit.edu algorithms to identify the five most likely off-target sites:

| sgRNA off-target primers | sequence | SEQ ID NO: |
|---|---|---|
| sgRNA #3 off-target Hit 1 F | TGATGGCTGGAGGGTAGAGG | 11 |
| sgRNA #3 off-target Hit 1 R | GGGTAGCTTAGTAGGGCTGC | 12 |
| sgRNA #3 off-target Hit 2 F | AAAACAACAACTCAGTCAAAATGCC | 13 |
| sgRNA #3 off-target Hit 2 R | TCCTCGCTGATCCAGAAGTT | 14 |
| sgRNA #3 off-target Hit 3 F | CAGTGGCAGGAGTACAAAGACAT | 15 |
| sgRNA #3 off-target Hit 3 R | ACTGTGGAGTTCATAGTCAAGGTC | 16 |
| sgRNA #3 off-target Hit 4 F | CATTGCACAAATCCGCCCTG | 17 |
| sgRNA #3 off-target Hit 4 R | TAAAAAGGGCTCCCTCGACAC | 18 |
| sgRNA #3 off-target Hit 5 F | CCCTCAAGCATGCAGGTACA | 19 |
| sgRNA #3 off-target Hit 5 R | GCAATCCTCTGAGTTGGGCT | 20 |
| sgRNA #1 off-target Hit 1 F | GCATGCAAGAGGAGCTCTGA | 21 |
| sgRNA #1 off-target Hit 1 R | CACAAGGCCTCTCACACCAT | 22 |
| sgRNA #1 off-target Hit 2 F | CTGGTAAGTGGAGGTGCTGG | 23 |
| sgRNA #1 off-target Hit 2 R | AGTGAGCTACAGCACAGCAG | 24 |
| sgRNA #1 off-target Hit 3 F | ACTGATCCAGCACGTAAGCC | 25 |
| sgRNA #1 off-target Hit 3 R | ATCTGTGTGTGCCCCAGAAG | 26 |
| sgRNA #1 off-target Hit 4 F | AGGAGGGGCTCTGTTCTCAT | 27 |
| sgRNA #1 off-target Hit 4 R | CTCACTCTTGCTCAGCCTGG | 28 |
| sgRNA #1 off-target Hit 5 F | TGCATTTCAAGGCAAGGCAG | 29 |
| sgRNA #1 off-target Hit 5 R | TAGGGGGCTTGGGACTGAG | 30 |

After amplification, PCR products were isolated via agarose gel electrophoresis and purified using a Zymoclean Gel DNA Recovery Kit (Zymo Research). Purified PCR fragments were submitted to Quintara Biosciences for Sanger sequencing. Sequence information was used to identify clones with the proper genetic modification. A homozygous clone for the gene insert was selected that had a normal karyotype, a normal genotype, and no off-target effects. Further, clones comprising heterozygous gene insertions were also identified and retained.

Cellular Cardiomyocyte Differentiation

The ES-Kir2.1 and iPS-Kir2.1 cell lines were cultured in feeder-free media and prepared for differentiation as previously described with modifications (see, e.g., Lian et al. (2012) “Robust cardiomyocyte differentiation from human pluripotent stem cells via temporal modulation of canonical Wnt signaling” Proc Natl Acad Sci USA 109: E1848-57; Lian et al. (2013) “Directed cardiomyocyte differentiation from human pluripotent stem cells by modulating Wnt/6-catenin signaling under fully defined conditions” Nature Protocols 8: 162-75, each of which is incorporated herein by reference in its entirety). In particular, five days prior to differentiation (“Differentiation Day −5”), cells were harvested and seeded. Media was removed from the stem cell cultures and the cells were and washed with 1 mL DPBS per well. Next, 1 mL of Versene was added to each well and the cells were incubated at 37° C. for 5 minutes. Cells were transferred to a 50-ml tube, an equal volume of medium was added, and the cells were resuspended by gentle pipetting to singularize the cells. Cells were counted, pelleted, and resuspended in culture medium (e.g., mTeSR1, Stem Flex, etc.) to provide cells at a concentration that is pipetted into 6-well plates at approximately 2 million cells per 6-well plate. Next, 10 μM ROCK inhibitor is added and the cell suspension is added to 6-well plates (e.g., Matrigel-coated 6-well plates, Senthemax-coated 6-well plates, etc.) and the plates are gently shaken to distribute cells evenly in the well.

During the four days prior to differentiation (“Differentiation Day −4 to −1”), cells were fed with 2.5 ml medium per well daily or every other day until cells were at or over 100% confluency. During the development of embodiments of the technology described herein, experiments were conducted during which data were collected indicating that the higher nutrient of Stem Flex relative to mTeSR1 allows for changing growth media every other (rather than every day), which reduces cell stress. Accordingly, in some embodiments, the methods comprise use of Stem Flex media instead of mTeSR1 due to the high metabolic requirements of the cells.

For differentiation, cells were confirmed to be free of spontaneous differentiation and were used at 100% confluency. To initiate differentiation on “Day 0”, culture medium was removed and replaced with 2.5 mL RPMI/B27-insulin, 12 μM CHIR99021, and 1 μg/mL insulin per well. During the development of embodiments of the technology described herein, experiments were conducted during which data were collected indicating that CHIR99021 sometimes kills all or most of the cells. Accordingly, in some embodiments, culture medium was removed and replaced with 2.5 mL RPMI/B27-insulin, 1 μg/mL insulin, and a lower amount of CHIR99021 (e.g., 6 μM) per well. Further, During the development of embodiments of the technology described herein, experiments were conducted during which data were collected indicating insulin could be omitted at this step. The data indicated that omitting insulin did not affect differentiation of the ES cells, but omitting the insulin affected differentiation of the iPS cells.

On the next day (“Day 1”), media was removed and replaced with RPMI/B27-insulin in each well. On Day 2, the medium was not changed. On Day 3 (e.g., approximately 72 hours after addition of CHIR99021 to inhibit WNT signaling), half of the medium (approximately 1.5 mL) was collected from each well. The collected medium was combined with the same volume of fresh RPMI/B27-insulin medium supplemented with 7.5 μM IWP2. The remaining 1.5 mL of medium was aspirated from each well of the 6-well plate and the new combined medium containing fresh RPMI/B27-insulin and IWP2 was added to each well. On Day 4, medium was not changed. On Day 5, media was removed and replaced with RPMI/B27-insulin in each well. On Day 6, medium was not changed. On Day 7 of differentiation and every 2 days thereafter, the medium is removed (e.g., by aspiration) and 2.5 mL RPMI/B27+ insulin medium is added.

For storage, SC derived cardiomyocytes (SC-CMs) were frozen between 14-16 days and subsequently thawed when needed. Thawed cells were purified using lactate media containing RPMI (glucose-fee)/B27+ insulin supplemented with sodium DL-lactate (19). After day 30, cells were used for imaging or electrophysiology experiments.

During the development of the technology described herein, multiple types of SC-CMs comprising an inducible I_{K1} and/or inducible potassium inward rectifier channel, e.g., a Kir (e.g., a Kir1, Kir2, Kir3, Kir4, Kir5, Kir6, or Kir7 (e.g., a Kir2.1, Kir2.2, Kir2.3, or Kir2.4 (e.g., a Kir2.1))) potassium inward rectifier channel were produced, isolated, characterized, and stored for use. For example, iPS SC-CM comprising a homozygous inducible gene insertion; iPS SC-CM comprising a heterozygous inducible gene insertion; ES SC-CM comprising a homozygous inducible gene insertion; and ES SC-CM comprising a heterozygous inducible gene insertion were produced as described herein.

Immunocytochemistry

SC-CMs were analyzed by immunocytochemistry as previously described (9). SC-CM with and without I_{K1}-induction were plated on coverslips and fixed using 4% formaldehyde, permeabilized with 0.1% Triton X-100, and blocked with 5% normal goat serum for 1 hour at room temperature. Fixed cells were incubated overnight with primary antibodies (e.g., one or more of anti-myosin light chain 2a (MLC2a), anti-Kir2.1, anti-cardiac troponin T (cTnT)) and DAPI stain at 4° C. Following washing with PBS-T (PBS containing 0.05% Tween 20), cells were incubated with secondary antibodies for one hour. Coverslips were then washed with PBS-T and mounted using Prolong gold-containing DAPI and imaged under a Leica confocal microscope after 24 hours.

Cellular Electrophysiology

SC-CMs were singularized and plated before cellular electrophysiology experiments on 12-mm poly-d-lysine/laminin or SyntheMax pre-coated coverslips. I_{K1} was induced by treating singularized cells with doxycycline (2 μM) (Thermo Fisher Scientific) for 48-72 hours prior to cellular electrophysiology analysis. Borosilicate glass pipettes (3-4 MΩ) were used (Model P-97; Sutter Instruments, Novato, Calif.). Whole cell capacitance was calculated by using the time domain technique (20).

Inward Rectifier Potassium Current.

I_{K1} was recorded by voltage-clamp using an Axopatch 200B amplifier and pCLAMP 10 (Molecular Devices, Sunnyvale, Calif.) at room temperature. The bath solution comprised 148 mM NaCl, 5.4 mM KCl, 1.0 mM MgCl₂, 1.8 mM CaCl₂, 0.4 mM NaH₂PO₄, 5.5 mM glucose, and 15 mM HEPES (pH 7.4, NaOH). The pipette filling solution comprised 150 mM K-gluconate, 5 mM EGTA, 10 mM HEPES, and 5 mM MgATP (pH 7.2, KOH). Calcium currents and calcium-sensitive chloride currents were blocked with nifedipine (5 μmol/L) in the bath solution. I_{K1} was recorded using a ramp protocol from a holding potential of −50 mV with a velocity of 25 mV/s between −120 to 20 mV.

Action Potentials

AP were measured under current clamp at 32° C. using an Axopatch 200B amplifier and pCLAMP 10 (Molecular Devices, Sunnyvale, Calif.). The bath solution comprised 148 mM NaCl, 5.4 mM KCl, 1 mM MgCl₂, 1.8 mM CaCl₂, 0.4 mM NaH₂PO₄, 5.5 mM glucose, and 15 mM HEPES (pH 7.4, NaOH). The pipette solution comprised 150 mM K-gluconate, 5 mM EGTA, 10 mM HEPES, and 5 mM MgATP (pH 7.2, KOH). Myocytes were paced at 0.5, 1, 2, and 3 Hz with a depolarizing pulse from a programmable digital stimulator (D55000; WPI, Sarasota, Fla.). AP properties including AP amplitude; resting membrane potential; AP duration at 10% (APD₁₀), 50% (APD₅₀), 70% (APD₇₀), and 90% (APD₉₀) of repolarization; and maximum upstroke velocity (dV/dtmax) were measured (pCLAMP 10; Matlab 6.0, Natick, Mass.). After the baseline recordings were made, di-LQTS was mimicked using either the hERG channel blocking drug E4031 (20 nM, 100 nM) (Alamone Labs) or late sodium current agonist ATX-II (30 nM) (Alamone Labs). Myocytes were paced at 0.5 and 1 Hz. Higher frequencies were not feasible due to AP prolongation. AP properties identified above were re-measured. EADs were occasionally induced and were evaluated for EAD take-off potential and EAD peak voltage.

Human iPSC Comprising an Inducible Kir2.1.

During the development of embodiments of the technology provided herein, experiments were conducted to produce human induced pluripotent stem cells comprising an inducible Kir2.1. An established human induced pluripotent stem cell (hiPSC; WiCell, Madison, Wis.; see, e.g., Yu et al. (2007) “Induced pluripotent stem cell lines derived from human somatic cells” Science 318: 1917-20, incorporated herein by reference) was modified using the CRISPR-Cas9 system to create iPSC comprising an inducible Kir2.1. A human cDNA clone of Kir2.1 as described above (e.g., comprising SEQ ID NO: 1) (see, e.g., Eckhardt et al. (2007) “KCNJ2 mutations in arrhythmia patients referred for LQT testing: a mutation T305A with novel effect on rectification properties. Heart Rhythm 4: 323-29, incorporated herein by reference) was introduced into the hiPSC using a doxycycline-inducible TRE3G reporter at the AAVS1 locus (see, e.g., Chen et al. (2015) “Engineering Human Stem Cell Lines with Inducible Gene Knockout using CRISPR/Cas9” Cell Stem Cell 17: 233-44, incorporated herein by reference). Genotyping was performed by selecting single-cell colonies and isolating genomic DNA using QuickExtract DNA Extraction Solution 1.0 (Epicentre, Madison, Wis.). All genotyping and subsequent sequencing was performed using Q5 polymerase-based PCR (New England BioLabs, Ipswich, Mass.). PCR products were purified using a Zymoclean Gel DNA Recovery Kit (Zymo Research, Irvine, Calif.) and submitted to Quintara Biosciences for Sanger sequencing to identify clones comprising the proper genetic modification. Using the five highest-likelihood off-target sites predicted by the crispr.mit.edu algorithms, off-target analysis was done to identify non-specific genome editing produced by the CRISPR-Cas9 system. Four clones with the proper genetic modification were selected. All clones lacked off-target effects, have a normal karyotype, and normal genotype analysis.

hiPSC-Cardiomyocyte Differentiation

hiPSCs were cultured in mTeSR1 media (WiCell) or StemFlex (Thermo Fischer Scientific) on Matrigel (GFR, Corning) coated 6-well plates. Differentiation of hiPSCs to cardiomyocytes (hiPSC-CMs) was performed using the small molecule GiWi protocol as previously described (see, e.g., Lian et al. (2012) “Robust cardiomyocyte differentiation from human pluripotent stem cells via temporal modulation of canonical Wnt signaling” Proceedings of the National Academy of Sciences of the United States of America 109: E1848-57; and Lian (2013) “Directed cardiomyocyte differentiation from human pluripotent stem cells by modulating Wnt/beta-catenin signaling under fully defined conditions” Nat Protoc 8: 162-75, each of which is incorporated herein by reference). hiPSC-CMs were frozen at 14 to 16 days of differentiation and thawed when needed. Thawed cells were purified using lactate media (see, e.g., Tohyama et al. (2013) “Distinct metabolic flow enables large-scale purification of mouse and human pluripotent stem cell-derived cardiomyocytes” Cell Stem Cell 12: 127-37, incorporated herein by reference) containing RPMI (glucose-fee) plus B27 complete supplement and further supplemented with sodium DL-lactate (Sigma). hiPSC-CMs were purified in the lactate media for 10 days.

Iₖ₁ Induction in hiPSC-Cardiomyocytes

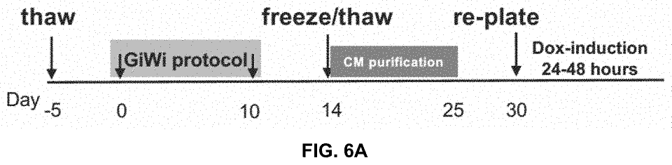

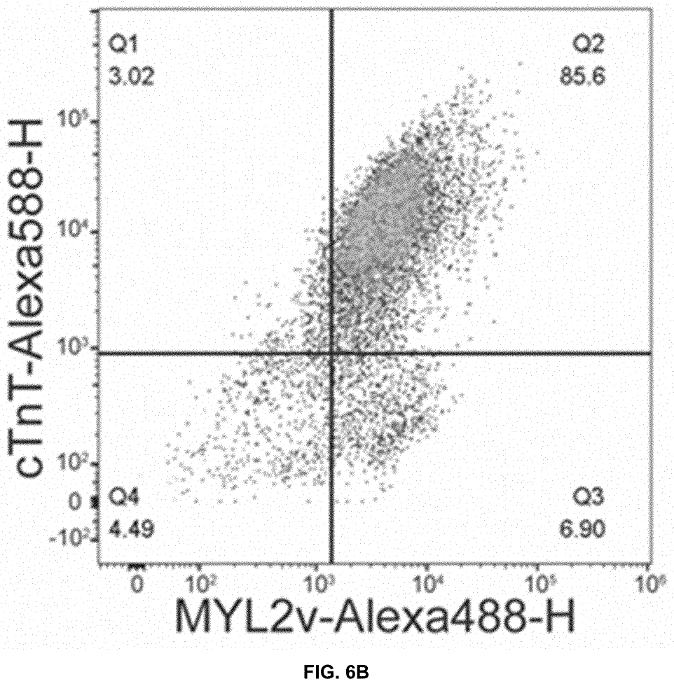

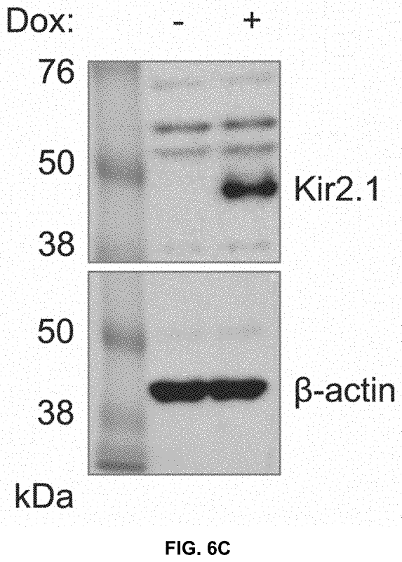

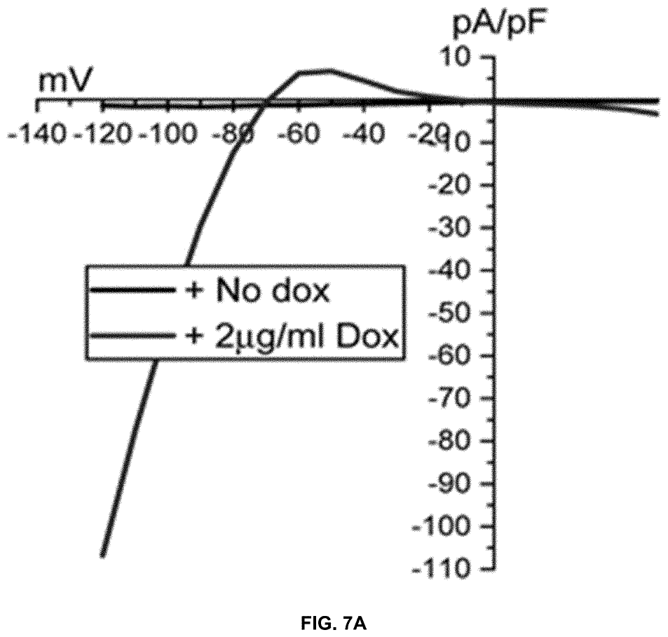

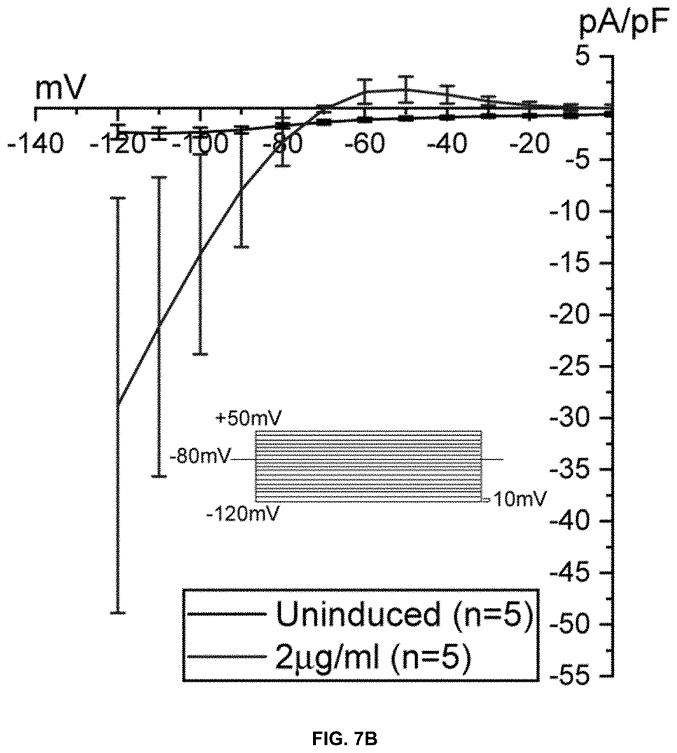

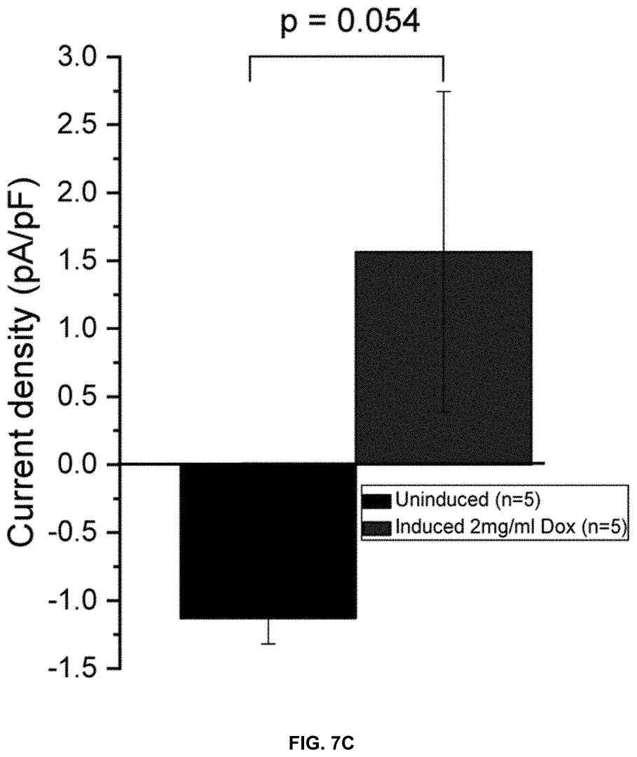

For I_{K1} induction, doxycycline (1-2 μg/ml) (Thermo Fisher Scientific, Waltham, Mass.) was added to the 30-day hiPSC-CMs in culture. After 24 to 48 hours, induced hiPSC-CMs were used for Western blot, flow cytometry, or electrophysiology experiments at days 33-35. FIG. 6A is a schematic drawing showing methods for differentiation and doxycycline induction. For rigorous analysis, several clones were carried forward for differentiation into CMs and I_{K1} quantified for in all lines (FIG. 7).

Western Blot Analysis of hiPSC-Cardiomyocytes

An equal number of hiPSC-CMs with and without I_{K1}-induction (2 mg/ml doxycycline for 48 hours) were lysed with NP40 lysis buffer containing a protease inhibitor cocktail. Insoluble debris was pelleted, and the soluble fraction was separated by SDS-PAGE and transferred to a PVDF membrane. Kir2.1 was detected using an anti-Kir2.1 antibody (Santa Cruz Biotechnology, Dallas, Tex.) followed by an HRP-conjugated secondary antibody. The membrane was then stripped and re-probed for a loading control using a mouse anti-beta-actin antibody (Abcam) followed by an HRP-conjugated secondary antibody. Proteins were detected using an ECL kit (Thermo Scientific Pierce).

Flow Cytometry of hiPSC-Cardiomyocytes

hiPSC-cardiomyocytes were singularized with TrypLE Express (Thermo Fisher). Singularized cells were fixed with 1% paraformaldehyde at 37° C. for 10 minutes, permeabilized, and stained with the primary antibodies MLC2v (ProteinTech; Cat #10906-1-AP) and cTnT (Thermo Fisher; Cat # MS-295-P) in FACS buffer (PBS containing 0.5% BSA, 0.1% NaN₃ and 0.1% Triton). A 1:11000 dilution of the secondary antibodies AlexaFluor 488 and 568 (Thermo Fisher) were added. All Data were collected on a Thermo Fisher Attune NxT Cytometer.

Cellular Electrophysiology of hiPSC-Cardiomyocytes

hiPSC-CMs were split and plated onto pre-coated coverslips with Synthemax (Sigma-Aldrich, St. Louis, Mo.). I_{K1} was induced in cells on coverslips with doxycycline (2 μM) (Thermo Fisher Scientific) for 48-72 hours before cellular electrophysiology analysis. Borosilicate glass pipettes (3-4 MΩ) were used (Model P-97; Sutter Instruments, Novato, Calif.). Whole cell capacitance was calculated using the time domain technique (see, e.g., Lindau and Neher (1988) “Patch-clamp techniques for time-resolved capacitance measurements in single cells. Pflugers Arch 411: 137-46, incorporated herein by reference). I_{K1} was recorded by voltage-clamp using an Axopatch 200B amplifier and pCLAMP 10 (Molecular Devices, Sunnyvale, Calif.) at room temperature. The bath solution comprised 148 mM NaCl, 5.4 mM KCl, 1.0 mM MgCl₂, 1.8 mM CaCl₂, 0.4 mM NaH₂PO₄, 5.5 mM glucose, and 15 mM HEPES (pH 7.4, NaOH). The pipette filling solution comprised 150 mM K-gluconate, 5 mM EGTA, 10 mM HEPES, and 5 mM MgATP (pH 7.2, KOH). Calcium currents and calcium-sensitive chloride currents were blocked with nifedipine (5 μmol/l in the bath solution. I_{K1} was recorded using a step protocol from a holding potential of −80 mV using sequential 10-mV steps from −120 mV to +50 mV in 100-ms steps. Following the recording, cells were perfused with bath solution containing 0.5 mM barium chloride and the I-V protocol was repeated and barium subtracted currents were calculated. Currents are given normalized to cell capacitance.

Statistics

All data are presented as mean SE. Statistical comparisons were carried out using Student's unpaired t-test or ANOVA, using OriginLab (Northampton, Mass.) or Microsoft Excel software.

### Example 1—Immunostaining of SC-CM

During the development of embodiments of the technology described herein, experiments were conducted to assess characteristics and cellular morphology of SC-CM expressing Kir2.1 relative to SC-CM not expressing Kir2.1. In particular, SC-CMs as described herein were induced to express Kir2.1 and stained with anti-myosin light chain 2a (MLC2a) antibody, anti-Kir2.1 antibody, anti-cardiac troponin T (cTnT) antibody, and with DAPI (FIG. 1, bottom row). Non-induced SC-CMs were used as a control (FIG. 1, top row). The images indicated that cell morphology and staining for MLC2a and cTnT were similar, but indicated an increased organization of myofibrils in the induced cells, similar to previous reports (9). Non-induced cells, consistent with other SC-CM, were observed to have low or undetectable Kir2.1 expression. In contrast, Kir2.1-induced cells demonstrated strong Kir2.1 staining. These results indicate that the induced and non-induced cells differentiate normally into cardiomyocytes and no un-intended expression of the gene insert occurs (e.g., in the non-induced cells).

### Example 2—I_{K1} Measured in Kir2.1 Induced Cardiomyocytes

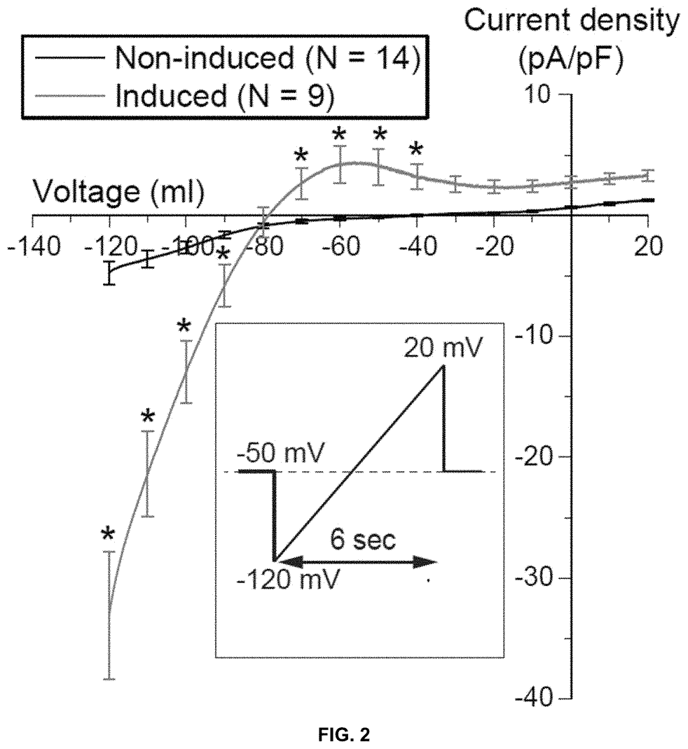

During the development of embodiments of the technology described herein, experiments were conducted to measure the I_{K1} inward rectifier current in SC-CM induced to express Kir2.1. Conventional SC-CMs spontaneously contract and have a small or non-detectable endogenous I_{K1} density (5, 6, 9). Embodiments of the SC-CMs described herein were detected to have similarly small endogenous or undetectable I_{K1} when not induced (FIG. 2, black trace). Following Kir2.1 induction, SC-CMs became quiescent.

Data were collected measuring the summary current density from the SC-CMs with induced I_{K1} (FIG. 2) using a voltage ramp protocol as shown in the inset of FIG. 2. The peak inward current density at −120 mV was −33.11±5.26 pA/pF and peak outward current density at −60 mV was 4.25±1.53 pA/pF. The maximum inward and outward current densities of I_{K1}-induced SC-CMs are similar in magnitude to reports of endogenous I_{K1} in adult human myoctyes (12). These data accord with the immunostaining data indicating strong and physiologic levels of Kir2.1 following the induction protocol.

### Example 3—AP Characteristics of I_{K1}-Induced SC-CMs

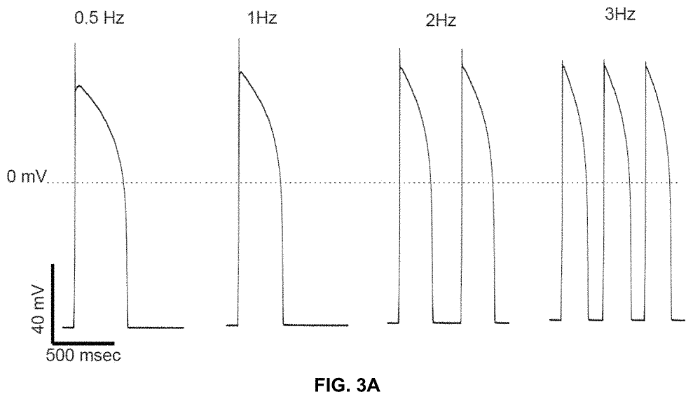

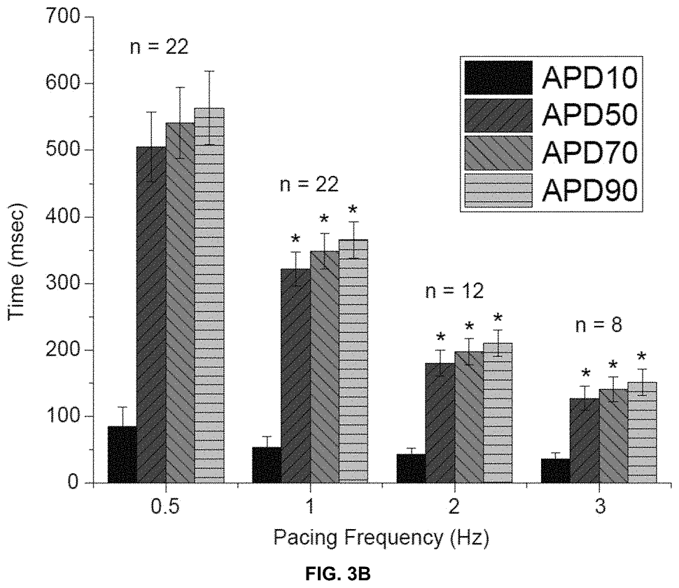

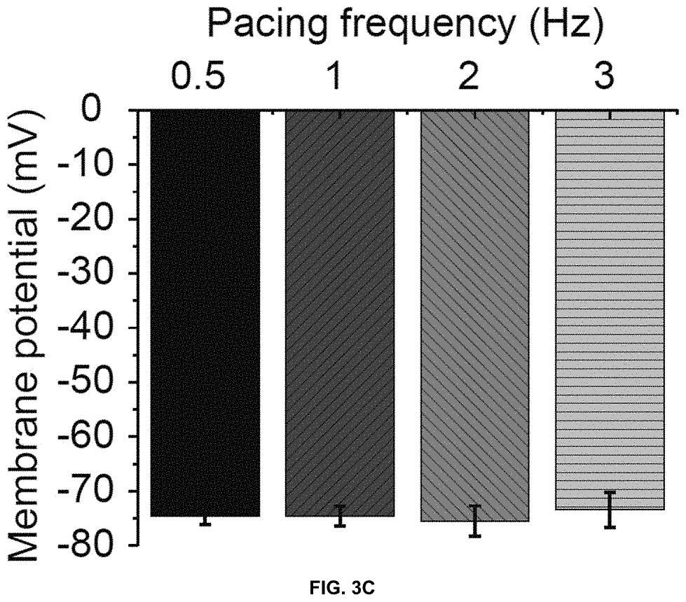

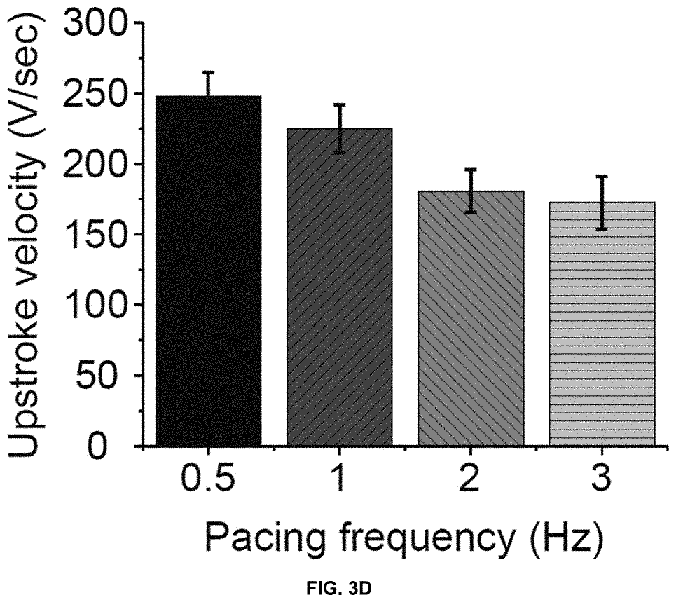

During the development of embodiments of the technology described herein, experiments were conducted to measure APs from I_{K1}-induced SC-CMs. During the experiments, APs from I_{K1}-induced SC-CMs were measured and identified as being atrial-like or ventricular-like APs based on AP duration at 5% of repolarization (APD₅). The cardiomyocytes exhibiting atrial-like APs were not studied further in this report. The AP characteristics of ventricular-like cardiomyocytes were measured (FIGS. 3B, 3C, and 3D) at pacing frequencies of 0.5, 1.0, 2.0, and 3.0 Hz applied as shown in FIG. 3A. Values for RMP, dV/dT max, APD₁₀, APD₅₀, APD₇₀ and APD₉₀ are tabulated in Table 2 at these pacing frequencies.

TABLE 2
APs of ventricular-like cardiomyocytes

| | RMP (mV) | dV/dT max (V/s) | APD₁₀ (ms) | APD₅₀ (ms) | APD₇₀ (ms) | APD₉₀ (ms) |
|---|---|---|---|---|---|---|
| 0.5 Hz | −75.13 ± 1.61 | 247.61 ± 17.10 | 85.53 ± 28.25 | 505.1 ± 52.09 | 541.21 ± 53.28 | 563.39 ± 54.79 |
| 1.0 Hz | −75.08 ± 1.80 | 224.86 ± 17.10 | 53.75 ± 16.5 | 321.65 ± 25.49 | 348.38 ± 26.44 | 365.38 ± 27.67 |
| 2.0 Hz | −76.35 ± 2.86 | 180.56 ± 15.04 | 43.58 ± 9.77 | 180.41 ± 19.50 | 197.95 ± 19.39 | 210.46 ± 19.39 |
| 3.0 Hz | −73.44 ± 3.20 | 172.42 ± 18.80 | 36.42 ± 9.04 | 127.14 ± 18.02 | 140.56 ± 18.46 | 151.44 ± 19.44 |

The resting membrane potential (RMP) and dV/dt max did not vary significantly with different pacing frequencies (Table 2; FIG. 3C and FIG. 3D, respectively). These data indicate that the RMP is more negative than reported previously for SC-CMs and is normally polarized close to the potassium reversal potential, consistent with adult cardiomyocytes. The dV/dt max values are similar to those found in human adult cardiomyocytes, but higher than reported for SC-CMs. FIG. 3B summarizes the data collected for ventricular-like SC-CMs showing APD rate adaptation with pacing frequencies of 0.5, 1.0, 2.0, and 3.0 Hz. As is consistent with adult ventricular myocytes, ventricular-like cardiomyocyte APD shortened progressively as the pacing rate increases. Our results highlight the physiologic range and rate dependency of APs. The dV/dtmax in ventricular-like cardiomyocytes is a measure of sodium channel availability and the data collected during experiments described herein have values that are similar to values measured for human adult cardiomyocytes and that are higher than reported for previous hPSC-CMs (see, e.g., Ma et al. (2011) “High purity human-induced pluripotent stem cell-derived cardiomyocytes: electrophysiological properties of action potentials and ionic currents” Am J Physiol Heart Circ Physiol 301: H2006-17; and Koumi et al. (1995) “beta-Adrenergic modulation of the inwardly rectifying potassium channel in isolated human ventricular myocytes. Alteration in channel response to beta-adrenergic stimulation in failing human hearts” J Clin Invest 96: 2870-81, each of which is incorporated herein by reference). Furthermore, the AP traces for I_{K1}-induced SC-CMs according to the technology described herein displayed a “spike-and-dome” shape with a stable, well defined plateau phase, which are AP characteristics of mature adult cardiomyocytes (see, e.g., FIG. 3A) (e.g., due to the presence of the Ca²⁺-independent transient outward K⁺ current (I_{TO}) current).

### Example 4—Cardiomyocytes Model Drug-Induced Long QT Syndrome

In some embodiments, the technology provides a cell line to model di-LQTS in a SC-CM platform. Accordingly, during the development of embodiments of the technology described herein, experiments were conducted to study the response of the SC-CMs with enhanced I_{K1} to AP-(QT interval) prolonging medications. E4031 decreases Ix, by preferential block of hERG channels and its effects on APD and EAD responses have been evaluated in isolated cardiomyocytes (7, 11). While SC-CMs show AP prolongation in response to E4031, typical SC-CM may cease to show normal repolarization capability and fail to elicit a normal AP response at doses that elicit EADs (11). I_{K1} is essential for controlling cellular automaticity, but it is also essential for repolarization.

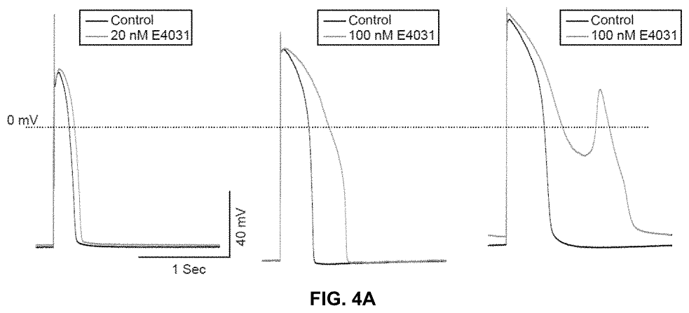

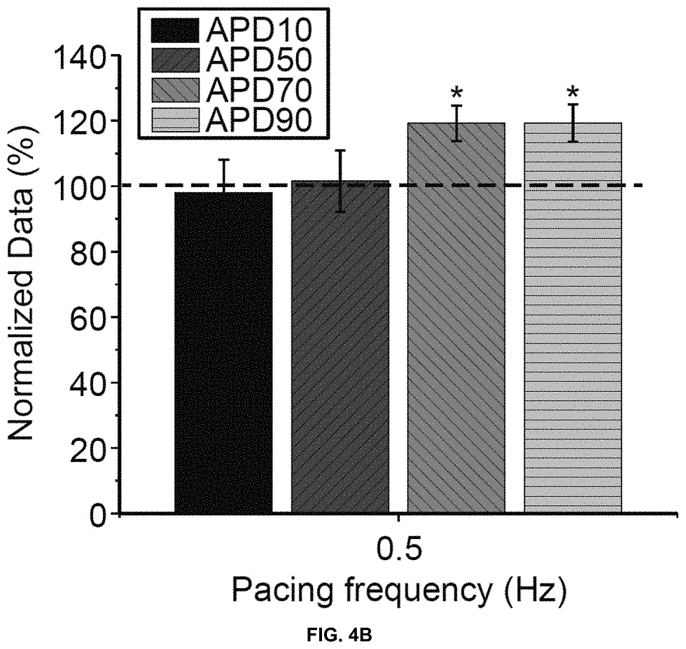

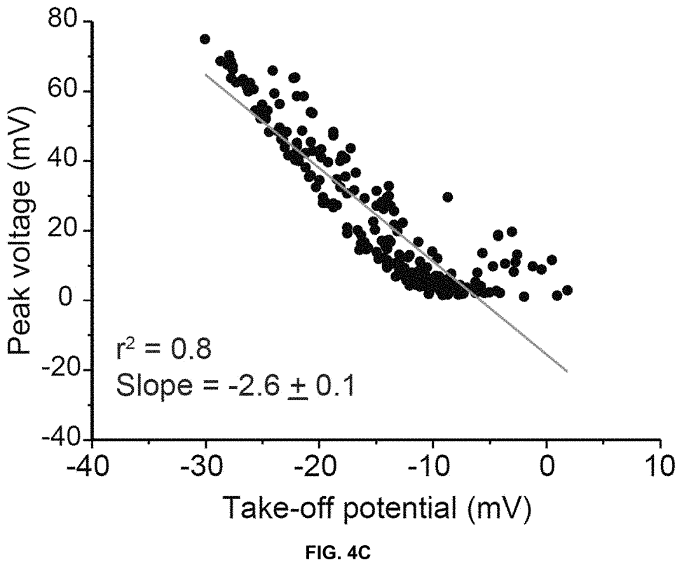

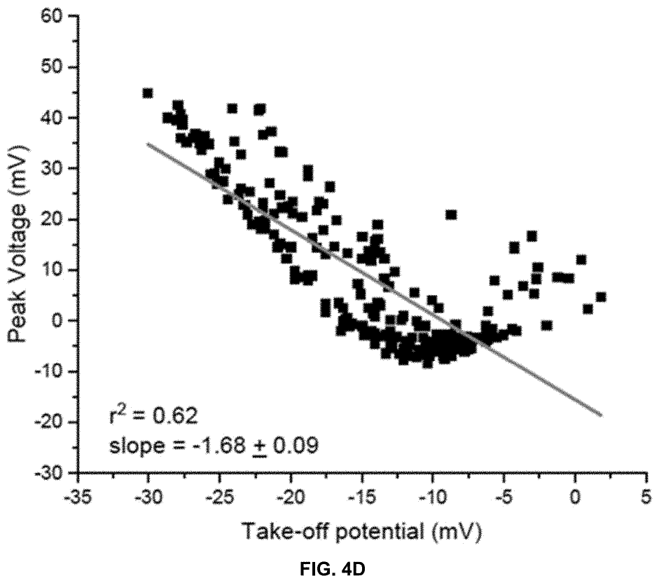

During the development of embodiments of the technology described herein, experiments were conducted, experiments were conducted in which E4031 (e.g., at 20 nM and 100 nM) was used to treat embodiments of I_{K1}-enhanced SC-CMs according to the technology described herein. FIG. 4A shows representative APs paced at 0.5 Hz. Compared to control data, E4031 exposure rapidly caused dose-dependent AP prolongation and induced EADs. FIG. 4B shows data for the effects of the lower dose (20 nM) of E4031 on APD₁₀, APD₅₀, APD₇₀, and APD₉₀ (also shown in Table 3). Compared with control data, low dose E4031 significantly prolongs the APD₇₀ and APD₉₀ (p<0.01). No significant effect was observed in the APD₁₀ or APD₅₀. These data indicate that the assay is highly sensitive to Ix, block. Table 3 shows the resting membrane potential (RMP) and dV/dt max values.

TABLE 3
APD at 20 nM E4031

| 0.5 Hz Pacing | RMP (mV) | dV/dTmax (V/s) | APD₁₀ (ms) | APD50 (ms) | APD70 (ms) | APD90 (ms) |
|---|---|---|---|---|---|---|
| Control | −73.4 ± 9.18 | 198.4 ± 17.62 | 190.64 ± 73.7 | 665.9 ± 128.31 | 706.8 ± 130.83 | 730.9 ± 133.67 |
| E4031 | −70.6 ± 4.11 | 207.5 ± 26.11 | 131.1 ± 53.4 | 633.8 ± 153.2 | 767.5 ± 164.8 | 802.4 ± 172.0 |
| Control | −76.2 ± 3.11 | 297.6 ± 33.38 | 27.3 ± 26.6 | 456.4 ± 74.6 | 497.9 ± 76.6 | 526.3 ± 80.0 |
| ATX-II | −76.2 ± 3.65 | 207.9 ± 39.55 | 45.2 ± 25.0 | 869.4 ± 111.7 | 1025.6 ± 107.4 | 1056.9 ± 106.5 |

The dV/dt max values are similar to those given in Table 2, were not affected by drug exposure, and are much larger than those reported from studies of conventional SC-CMs (5). Likewise, RMP remains normally polarized and stable despite E4031 treatment. EADs occurred in 2/2 cells treated with 100 nM E4021. Analysis of the AP plateau EAD take-off potential vs. peak EAD voltage is shown in FIG. 4C and FIG. 4D (two independent experiments). The data collected were fit as a linear regression. In parallel with prior studies, EADs demonstrated a steep negative slope of −2.6±0.1 (FIG. 4C) and −1.68±0.09 (FIG. 4D) with the highest peak voltage correlating with more negative take-off potential (8).

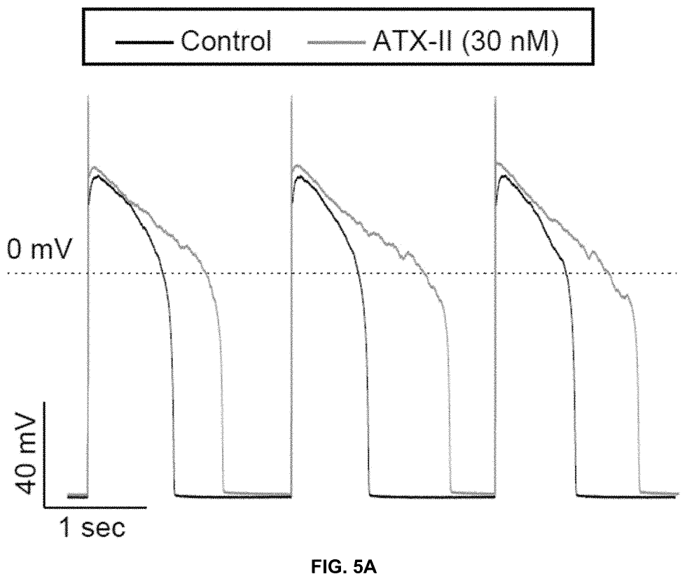

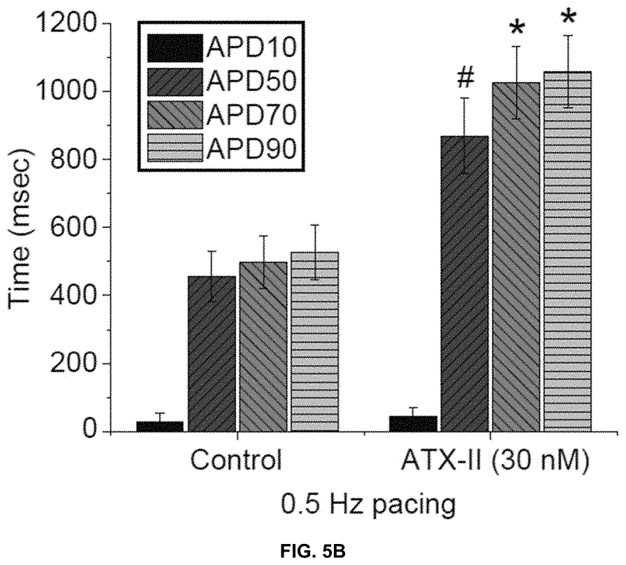

Further, during the development of embodiments of the technology described herein, experiments were conducted to analyze the effect of ATX-II, a late I_{Na} enhancing drug (delayed channel inactivation), on I_{K1}-induced SC-CMs. When paced at 0.5 Hz with 30 nM ATX-II, APD markedly increased. As shown in the exemplary AP traces in FIG. 5A, the AP plateau voltage was more positive and demonstrated beat-to-beat variability in APD. In addition, the AP plateau voltage became noisy. EADs comparable to E4031 did not occur despite significant AP prolongation. The AP characteristics with ATX-II exposure are shown in Table 3 and FIG. 5B. As with E4031, there is no difference in RMP and dV/dt max at baseline compared to ATX-II. In contrast, beginning after APD₁₀, the data indicate a significant AP prolongation throughout repolarization.

### Example 5—Human Induced Pluripotent Stem Cells Comprising Inducible I_{K1}

During the development of embodiments of the technology described herein, experiments were conducted to produce a hiPSC cell line comprising an inducible I_{K1} current using CRISPR-Cas9 gene editing. Data collected during these experiments indicated that the experiments were successful. In particular, the data indicated successful genetic modification of a hiPSC line using a CRISPR-Cas9 gene editing system to introduce a doxycycline inducible I_{K1} current with stable Kir2.1 expression and without off-target effects. The schematic method for cardiomyocyte differentiation and IK1 induction is shown in FIG. 6A. Flow cytometry data collected during the development of embodiments of the technology indicated that the technology produced highly pure 30-day cardiomyocytes (FIG. 6B). In particular, greater that 85% of the hiPSC-CMs were identified as being positive for both cardiac troponin T (cTnT) and myosin light chain 2v (MLC2v) and greater than 95% of cells were identified as being positive for at least one of cardiac troponin T (cTnT) or myosin light chain 2v (MLC2v).

hiPSC-CM cell lysates were analyzed for Kir2.1 using Western blot techniques. Both the high purity and high percentage of ventricular myocytes are established properties of the GiWi protocol (see, e.g., Lian et al. (2013) “Directed cardiomyocyte differentiation from human pluripotent stem cells by modulating Wnt/beta-catenin signaling under fully defined conditions” Nat Protoc 8: 162-75; and Zhang et al. 92009) “Functional cardiomyocytes derived from human induced pluripotent stem cells. Circulation research 104: e30-41, each of which is incorporated herein by reference). Kir2.1 expression was not detected in non-induced hiPSC-CMAs (FIG. 6C, upper panel, “−” indicating doxycycline was absent). In contrast, robust expression of Kir2.1 was detected in induced hiPSC-CM (FIG. 6C, upper panel, “+” indicating doxycycline was present). In a parallel Western blot experiment, beta-actin was used as a loading control (FIG. 6C, lower panel).

In human myocytes, I_{K1} is hyperpolarization-activated and has an outward component at physiologic voltages that peaks at −60 mV and has a reversal potential close to the potassium equilibrium potential. Thus, at the completion of repolarization of each AP the outward K+ current becomes the factor that dominates the RMP in all cardiomyocytes (Vaidyanathan et al. (2016) “IK1-Enhanced Human Induced Pluripotent Stem Cell-Derived Cardiomyocytes: An Improved Cardiomyocyte Model to Investigate Inherited Arrhythmia Syndromes” Am J Physiol Heart Circ Physiol 310(11): H1611-21, incorporated herein by reference). Without induction, the endogenous I_{K1} density in the hiPSC-CM produced according to the technology described herein had similarly small (nearly undetectable) endogenous I_{K1} without a typical I_{K1} I-V relationship or reversal potential (FIG. 7A and FIG. 7B, black line), consistent with prior hiPSC-CMs (see, e.g., Doss et al. (2012) “Maximum diastolic potential of human induced pluripotent stem cell-derived cardiomyocytes depends critically on I(Kr)” PLoS One 7: e40288; Ma et al. (2011) “High purity human-induced pluripotent stem cell-derived cardiomyocytes: electrophysiological properties of action potentials and ionic currents” Am J Physiol Heart Circ Physiol 301: H2006-17; and Vaidyanathan et al. (2016) “IK1-Enhanced Human Induced Pluripotent Stem Cell-Derived Cardiomyocytes: An Improved Cardiomyocyte Model to Investigate Inherited Arrhythmia Syndromes” Am J Physiol Heart Circ Physiol 310(11): H1611-21, each of which is incorporated herein by reference). The summation of hiPSC-CM following induction is shown in FIG. 7B and the step protocol is shown in the figure inset. The maximum outward current at −60 mV for the hiPSC-CM is graphically shown in FIG. 7C. These data indicate that the cell lines described herein have robust, physiologic maximum inward and outward I_{K1} that is similar in magnitude to the endogenous I_{K1} in adult human myocytes (see, e.g., Koumi et al. (1995) “beta-adrenergic and cholinergic modulation of the inwardly rectifying K+ current in guinea-pig ventricular myocytes. J Physiol 486 (Pt 3): 647-59; and Jost et al. 2013 “Ionic mechanisms limiting cardiac repolarization reserve in humans compared to dogs” J Physiol 591: 4189-206, each of which is incorporated herein by reference).

### Example 6—Quantitative PCR of hiPSC and hESC Comprising Inducible I_{K1}

During the development of embodiments of the technology described herein, quantitative PCR was performed on the hiPSC and hESC comprising inducible I_{K1} described herein. hiPSC described herein were cultured in mTeSR1 media (WiCell) or StemFlex (Thermo Fischer Scientific) on Matrigel (GFR, Corning) coated 6-well plates. From a single clone, hESC described herein were harvested and lysed and RNA was isolated using standard manufacture instructions. qPCR was performed for Kir2.1/KCNJ2 (primary target) using the following primers:

Kir2.1 Q-1F
(SEQ ID NO: 31)
TCCGAGGTCAACAGCTTCAC
Kir2.1 Q-1R
(SEQ ID NO: 32)
TTGGGCATTCATCCGTGACA

Primers targeting the housekeeping genes GAPDH and beta-actin were used for controls.

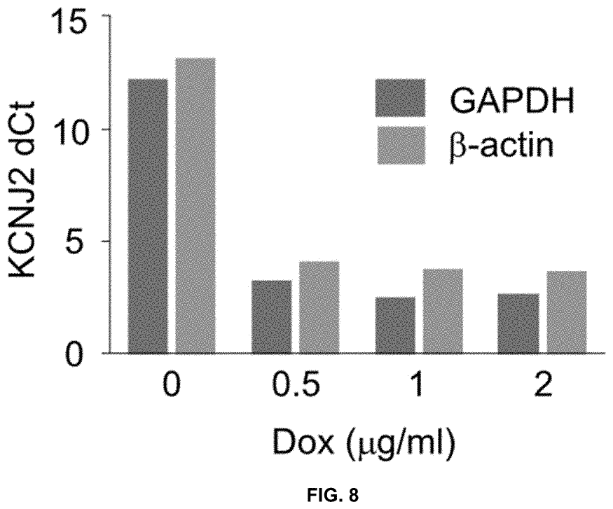

The cycle number at which point the target is detected is indicated by “Ct” with lower numbers indicating increased transcript and earlier detection. FIG. 8 provides the data from 3 separate experiments. The Ct value is normalized to the housekeeping genes, GAPDH and beta-actin, and expressed as dCt. Primer-dimer formation in the un-induced “0 dox” control caused false positive detection of signal at dCt of 10-15. The dCt for these samples is actually much higher, indicating significantly less transcript was present or possibly none at all. Therefore, no fold change was calculated relative to the uninduced “0 dox” control because it would not have been accurate. However, these data clearly indicate a significant increase in Kir2.1/KCNJ2 expression of mRNA with doxycycline induction.

### REFERENCES

1. Roden D M. Drug-induced prolongation of the QT interval. The New England journal of medicine. 2004; 350(10)1013-22.
2. Colatsky T, Fermini B, Gintant G, Pierson J B, Sager P, Sekino Y, Strauss D G, Stockbridge N. The Comprehensive in Vitro Proarrhythmia Assay (CiPA) initiative—Update on progress. J Pharmacol Toxicol Methods. 2016; 81:15-20.
3. Ferri N, Siegl P, Corsini A, Herrmann J, Lerman A, Benghozi R. Drug attrition during pre-clinical and clinical development; understanding and managing drug-induced cardiotoxicity. Pharmacol Ther. 2013; 138(3)470-84.
4. Goversen B, van der Heyden M A G, van Veen T A B, de Boer T P. The immature electrophysiological phenotype of iPSC-CMs still hampers in vitro drug screening; Special focus on I_{K1}. Pharmacol Ther. 2017.
5. Ma J, Guo L, Fiene S J, Anson B D, Thomson J A, Kamp T J, Kolaja K L, Swanson B J, January C T. High purity human-induced pluripotent stem cell-derived cardiomyocytes: electrophysiological properties of action potentials and ionic currents. Am J Physiol Heart Circ Physiol. 2011; 301(5):H2006-17.
6. Doss M X, Di Diego J M, Goodrow R J, Wu Y, Cordeiro J M, Nesterenko V V, Barajas-Martinez H, Hu D, Urrutia J, Desai M, Treat J A, Sachinidis A, Antzelevitch C. Maximum diastolic potential of human induced pluripotent stem cell-derived cardiomyocytes depends critically on I(Kr). PLoS One. 2012; 7(7); e40288.
7. Ando H, Yoshinaga T, Yamamoto W, Asakura K, Uda T, Taniguchi T, Ojima A, Shinkyo R, Kikuchi K, Osada T, Hayashi S, Kasai C, Miyamoto N, Tashibu H, Yamazaki D, Sugiyama A, Kanda Y, Sawada K, Sekino Y. A new paradigm for drug-induced torsadogenic risk assessment using human iPS cell-derived cardiomyocytes. J Pharmacol Toxicol Methods. 2017; 84:111-27.
8. January C T, Riddle J M, Salata J J. A model for early afterdepolarizations: induction with the Ca2+ channel agonist Bay K 8644. Circulation research. 1988; 62(3)563-71.
9. Vaidyanathan R, Markandeya Y S, Kamp T J, Makielski J C, Janaury C T, Eckhardt L L. I_{K1}-Enhanced Human Induced Pluripotent Stem Cell-Derived Cardiomyocytes: An Improved Cardiomyocyte Model to Investigate Inherited Arrhythmia Syndromes. Am J Physiol Heart Circ Physiol. 2016:ajpheart 00481 2015.
10. Lieu D K, Fu J D, Chiamvimonvat N, Tung K C, McNerney G P, Huser T, Keller G, Kong C W, Li R A. Mechanism-based facilitated maturation of human pluripotent stem cell-derived cardiomyocytes. Circ Arrhythm Electrophysiol. 2013; 6(1)191-201. Epub 2013/02/09.
11. Li M, Kanda Y, Ashihara T, Sasano T, Nakai Y, Kodama M, Hayashi E, Sekino Y, Furukawa T, Kurokawa J. Overexpression of KCNJ2 in induced pluripotent stem cell-derived cardiomyocytes for the assessment of QT-prolonging drugs. J Pharmacol Sci. 2017; 134(2)75-85.
12. Bett G C, Kaplan A D, Lis A, Cimato T R, Tzanakakis E S, Zhou Q, Morales M J, Rasmusson R L. Electronic “expression” of the inward rectifier in cardiocytes derived from human-induced pluripotent stem cells. Heart Rhythm 2013; 10(12)1903-10.
13. Konig S, Hinard V, Arnaudeau S, Holzer N, Potter G, Bader C R, Bernheim L. Membrane hyperpolarization triggers myogenin and myocyte enhancer factor-2 expression during human myoblast differentiation. J Biol Chem. 2004; 279(27):28187-96.
14. Thomson J A, Itskovitz-Eldor J, Shapiro S S, Waknitz M A, Swiergiel J J, Marshall V S, Jones J M. Embryonic stem cell lines derived from human blastocysts. Science. 1998; 282(5391):1145-7.
15. Eckhardt L L, Farley A L, Rodriguez E, Ruwaldt K, Hammill D, Tester D J, Ackerman M J, Makielski J C. KCNJ2 mutations in arrhythmia patients referred for LQT testing: a mutation T305A with novel effect on rectification properties. Heart Rhythm 2007; 4(3)323-9.
16. Chen Y, Cao J, Xiong M, Petersen A J, Dong Y, Tao Y, Huang C T, Du Z, Zhang S C. Engineering Human Stem Cell Lines with Inducible Gene Knockout using CRISPR/Cas9. Cell Stem Cell. 2015; 17(2)233-44.
17. Lian X, Hsiao C, Wilson G, Zhu K, Hazeltine L B, Azarin S M, Raval K K, Zhang J, Kamp T J, Palecek S P. Robust cardiomyocyte differentiation from human pluripotent stem cells via temporal modulation of canonical Wnt signaling. Proceedings of the National Academy of Sciences of the United States of America. 2012; 109(27):E1848-57.
18. Lian X, Zhang J, Azarin S M, Zhu K, Hazeltine L B, Bao X, Hsiao C, Kamp T J, Palecek S P. Directed cardiomyocyte differentiation from human pluripotent stem cells by modulating Wnt/beta-catenin signaling under fully defined conditions. Nat Protoc. 2013; 8(1):162-75.
19. Tohyama S, Hattori F, Sano M, Hishiki T, Nagahata Y, Matsuura T, Hashimoto H, Suzuki T, Yamashita H, Satoh Y, Egashira T, Seki T, Muraoka N, Yamakawa H, Ohgino Y, Tanaka T, Yoichi M, Yuasa S, Murata M, Suematsu M, Fukuda K. Distinct metabolic flow enables large-scale purification of mouse and human pluripotent stem cell-derived cardiomyocytes. Cell Stem Cell. 2013; 12(1)127-37.
20. Lindau M, Neher E. Patch-clamp techniques for time-resolved capacitance measurements in single cells. Pflugers Arch. 1988; 411(2)137-46.
21. Harrell M D, Harbi S, Hoffman J F, Zavadil J, Coetzee W A. Large-scale analysis of ion channel gene expression in the mouse heart during perinatal development. Physiol Genomics. 2007; 28(3)273-83.
22. Sacco S, Giuliano S, Sacconi S, Desnuelle C, Barhanin J, Amri E Z, Bendahhou S. The inward rectifier potassium channel Kir2.1 is required for osteoblastogenesis. Hum Mol Genet. 2015; 24(2)471-9.
23. Konig S, Beguet A, Bader C R, Bernheim L. The calcineurin pathway links hyperpolarization (Kir2.1)-induced Ca2+ signals to human myoblast differentiation and fusion. Development. 2006; 133(16):3107-14.
24. Goversen B, Becker N, Stoelzle-Feix S, Obergrussberger A, Vos M A, van Veen T A B, Fertig N, de Boer T P. A Hybrid Model for Safety Pharmacology on an Automated Patch Clamp Platform: Using Dynamic Clamp to Join iPSC-Derived Cardiomyocytes and Simulations of I_{K1} Ion Channels in Real-Time. Frontiers in Physiology 2018; 8: 1094.
25. Blinova K, Stohlman J, Vicente J, Chan D, Johannesen L, Hortigon-Vinagre M P, Zamora V, Smith G, Crumb W J, Pang L, Lyn-Cook B, Ross J, Brock M, Chvatal S, Millard D, Galeotti L, Stockbridge N, Strauss D G. Comprehensive Translational Assessment of Human-Induced Pluripotent Stem Cell Derived Cardiomyocytes for Evaluating Drug-Induced Arrhythmias. Toxicol Sci. 2017; 155(1)234-47.
26. Lathrop D A, Varro A, Nanasi P P, Bodi I I, Takyi E, Pankucsi C. Differences in the Effects of d- and dl-Sotalol on Isolated Human Ventricular Muscle: Electromechanical Activity After Beta-Adrenoceptor Stimulation. J Cardiovasc Pharmacol Ther. 1996; 1(1)65-74.
27. Studenik C R, Zhou Z, January C T. Differences in action potential and early afterdepolarization properties in LQT2 and LQT3 models of long QT syndrome. Br J Pharmacol. 2001; 132(1)85-92.
28. Xie Y, Sato D, Garfinkel A, Qu Z, Weiss J N. So little source, so much sink: requirements for afterdepolarizations to propagate in tissue. Biophys J. 2010; 99(5):1408-15.

All publications and patents mentioned in the above specification (both in the above References section and throughout the specification text) are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the technology as claimed should not be unduly limited to such specific embodiments. Indeed, various modifications of the described modes for carrying out the technology that are obvious to those skilled in the art are intended to be within the scope of the following claims.

We claim:

1. A stem cell derived cardiomyocyte comprising a nucleic acid encoding an inducible potassium inward rectifier channel.

2. The stem cell derived cardiomyocyte of claim 1 wherein said nucleic acid comprises a Kir sequence.

3. The stem cell derived cardiomyocyte of claim 1 wherein said nucleic acid comprises a Kir2 sequence.

4. The stem cell derived cardiomyocyte of claim 1 wherein said wherein said nucleic acid comprises a Kir2.1 sequence.

5. The stem cell derived cardiomyocyte of claim 1 comprising a Kir2.1 cDNA or genomic sequence.

6. The stem cell derived cardiomyocyte of claim 1 comprising a nucleic acid comprising an inducible promoter operably linked to a nucleic acid encoding a Kir2.1.

7. The stem cell derived cardiomyocyte of claim 1 comprising a doxycycline-inducible promoter operably linked to a nucleic acid encoding a Kir2.1.

8. The stem cell derived cardiomyocyte of claim 1 comprising a TRE3G promoter operably linked to a nucleic acid encoding a Kir2.1.

9. The stem cell derived cardiomyocyte of claim 1 wherein said nucleic acid comprises a sequence from KCNJ2.

10. The stem cell derived cardiomyocyte of claim 1 wherein said nucleic acid comprises a sequence that is at least 80% identical to a sequence from KCNJ2.

11. The stem cell derived cardiomyocyte of claim 1 wherein said nucleic acid comprises a sequence that is at least 90% identical to a sequence from KCNJ2.

12. The stem cell derived cardiomyocyte of claim 1 wherein said nucleic acid comprises a sequence that is at least 95% identical to a sequence from KCNJ2.

13. The stem cell derived cardiomyocyte of claim 1 wherein said nucleic acid comprises a sequence that is at least 99% identical to a sequence from KCNJ2.

14. A stem cell derived cardiomyocyte comprising an inducible potassium inward rectifier current (I_{K1}).

15. A method of producing a physiologically mature stem cell derived cardiomyocyte, the method comprising:
a) providing a stem cell derived cardiomyocyte comprising a nucleic acid encoding an inducible potassium inward rectifier channel; and
b) inducing expression of said inducible potassium inward rectifier channel in said stem cell derived cardiomyocyte.

16. The method of claim 15 further comprising pacing said stem cell derived cardiomyocyte.

17. The method of claim 15 wherein said inducing comprises contacting said stem cell derived cardiomyocyte comprising a nucleic acid encoding an inducible potassium inward rectifier channel with a composition comprising an inducer.

18. The method of claim 15 wherein said providing comprises thawing a stem cell derived cardiomyocyte comprising a nucleic acid encoding an inducible potassium inward rectifier channel from a stored preparation.

19. The method of claim 15 wherein said providing comprises constructing said cardiomyocyte comprising a nucleic acid encoding an inducible potassium inward rectifier channel using a CRISPR technology.

20. A system for testing the cardiac safety of a drug, the system comprising:
i) a stem cell derived cardiomyocyte expressing a potassium inward rectifier channel; and
ii) a cellular electrophysiology measurement system.

21. The system of claim 20 further comprising said drug.

22. The system of claim 20 further comprising an inducer composition for inducing expression of said potassium inward rectifier channel in said stem cell derived cardiomyocyte.

23. The system of claim 20 wherein stem cell derived cardiomyocyte has a physiologically mature phenotype.

24. The system of claim 20 further comprising a component to pace said stem cell derived cardiomyocyte.

25. A cell expressing an inducible potassium inward rectifier channel.

26. The cell of claim 25 wherein said cell is a muscle cell or a neurocyte.

27. The cell of claim 25 wherein said cell is a differentiated stem cell.

28. A composition comprising a cell expressing an inducible potassium inward rectifier channel.

29. The composition of claim 28 further comprising a test compound.

30. The composition of claim 28 further comprising an inducing compound.

31. A method for testing a compound for cardiac safety, the method comprising:
a) providing a physiologically mature stem cell derived cardiomyocyte comprising a nucleic acid encoding an inducible potassium inward rectifier channel;
b) contacting said physiologically mature stem cell derived cardiomyocyte with a test compound; and
c) measuring a physiological phenotype of said physiologically mature stem cell derived cardiomyocyte.

32. The method of claim 31 wherein said physiological phenotype is an action potential (AP), AP amplitude, resting membrane potential, AP duration at 10% of repolarization (APD10), AP duration at 50% of repolarization (APD50), AP duration at 70% of repolarization (APD70), AP duration at 90% of repolarization (APD90) of repolarization, or maximum upstroke velocity (dV/dtmax).

33. The method of claim 31 further comprising comparing the physiological phenotype of said physiologically mature stem cell derived cardiomyocyte in the presence and absence of said test compound.

34. The method of claim 31 wherein said physiologically mature stem cell derived cardiomyocyte has a potassium inward rectifier current similar to a cardiomyocyte in vivo.
